SEQUENCE LISTING (10 sequences)

SEQ ID NO: 1 - DNA, 488 bp, Homo sapiens
ccttgttccgtatttaaatcgctgtctccaagtcctttttccaccagctcagatagcacagactttaggcattcctcccactcagctctccaagtacggtaacctagggcatgtgaacatcggcgccattcaggagcccctcgcctttatcctgccaaagagagagacgcttttcaccctggatgaccaggcgctggggcccgagctcacagctccagcaccagagcctcccgccgaggagccacgcctggagcccgcgggcccagcctgcccggagggagggcgagcggagacgcaggccgaaccgcccagcgtggggccctagccggcgtcccctgcctccagaacgcgggctggaccccaatggagaacaggtggtgtggcaggcgagcgggtgggcagcccgcatcatccagcacgagatggaccacctgcagggctgcctgtttattgacaaaatggacagcaggacgttcacaaacgtctat

SEQ ID NO: 2 - protein, 163 aa, Homo sapiens
  VARIANT (1)...(163): Xaa = Any Amino Acid
LFRIXIAVSKSFFHQLRXHRLXAFLPLSSPSTVTXGMXTSAPFRSPSPLSCQRERRFSPWMMTRRWGPSSQLQHQSLPPRSHAWSPRAQPARREGERRRRPNRPAWGPSRRPLPPERGLDPNGEQVVWQASGWAARIIQHEMDHLQGCLFIDKMDSRTFTNVY

SEQ ID NO: 3 - DNA, 630 bp, Homo sapiens
  misc_feature (1)...(630): n = A,T,C or G
ttttcacatgcttacctgcatttttaaagacagctttcaggtatttggggactacattattaccaaaccttggctttgggagattatacaggtccgaggaactcgtgtctactgcagacgaatgcaattaccccaccttcctccatacagaattgttaggaaatgtccactcctttgggggtgatttttctcctcaagttgtagccaacattttgtccgtaactgatttcagggcaaacatttctgacatcttcctccagctcagtctgccatgccttggcaatccagtttcctgtcatatgcgagccatccaagttgatgccaagtaagatttgcccagctcaaagtgaaagtgtttgcgtcttggtatccggaatcctcagccccagtagcaaagctttagtcattcaccttcatccaatagacgtttgtgaacgtcctgctgtccattttgtcaataaacaggcagccctgcaggtggtccatctcgtgctggatgatgcgggctgcccacccgctcgcctgccacaccacctgttctccattggggtccagccctgagatctgcaccgnctggaagcggggcacgcangccangaaagccgcgacgctctcgcagccctcgggaaa

SEQ ID NO: 4 - protein, 210 aa, Homo sapiens
  VARIANT (1)...(210): Xaa = Any Amino Acid
FPEGCESVAAFXACVPRFQXVQISGLDPNGEQVVWQASGWAARIIQHEMDHLQGCLFIDKMDSRTFTNVYWMKVNDXSFATGAEDSGYQDANTFTLSWANLTWHQLGWLAYDRKLDCQGMADXAGGRCQKCLPXNQLRTKCWLQLEEKNHPQRSGHFLTILYGGRWGNCIRLQXTRVPRTCIISQSQGLVIMXSPNTXKLSLKMQVSMXK

SEQ ID NO: 5 - DNA, 732 bp, Homo sapiens
atggcccggctgtggggcgcgctgagtctttggccactgtgggcggccgtgccgtggggcggggcggcagccgtcggtgtccgggcttgcagctccacggccgccccggacggcgtcgagggcccggcgctgcggcgctcctattggcgccacctgaggcgtctggtgctgggtcctcccgaaccgccgttctcgcacgtgtgccaagtcggggacccggtgctgcgcggcgtggcggccccggtggagcgggcgcagctaggcgggcccgagctgcagcggctgacgcaacggctggtccaggtgatgcggcggcggcgctgcgtgggcctaagcgcgccgcagctgggggtgccgcggcaggtgctggcgctggagctccccgaggcgctgtgtcgggagtgcccgccccgccagcgcgcgctccggcaaatggagcccttccccctgcgcgtgttcgtgaaccccagcctgcgagtgcttgacagccgcctggtcacctttcccgagggctgcgagagcgtcgccggcttcctggcctgcgtgccccgcttccaggcggtgcagatctcagggctggaccccaatggagaacaggtggtgtggcaggcgagcgggtgggcagcccgcatcatccagcacgagatggaccacctgcagggctgcctgtttattgacaaaatggacagcaggacgttcacaaacgtctattggatgaaggtgaatgactaa

SEQ ID NO: 6 - protein, 243 aa, Homo sapiens
MARLWGALSLWPLWAAVPWGGAAAVGVRACSSTAAPDGVEGPALRRSYWRHLRRLVLGPPEPPFSHVCQVGDPVLRGVAAPVERAQLGGPELQRLTQRLVQVMRRRRCVGLSAPQLGVPRQVLALELPEALCRECPPRQRALRQMEPFPLRVFVNPSLRVLDSRLVTFPEGCESVAGFLACVPRFQAVQISGLDPNGEQVVWQASGWAARIIQHEMDHLQGCLFIDKMDSRTFTNVYWMKVND

SEQ ID NO: 7 - protein, 5 aa, Artificial Sequence
  zinc-dependent metalloprotease motif
HEXXH

SEQ ID NO: 8 - DNA, 24 bp, Homo sapiens
cagcgatttaaatacggaacaagg

SEQ ID NO: 9 - DNA, 24 bp, Homo sapiens
aaaatgcaggtaagcatgtgaaaa

SEQ ID NO: 10 - DNA, 24 bp, Artificial Sequence
  random oligonucleotide
tcaactgactagatgtacatggac

(19) United States
(12) Patent Application Publication
(10) Pub. No.: US 2003/0003488 A1
(43) Pub. Date: Jan. 2, 2003

(54) Regulation of human mitochondrial deformylase

(76) Inventor: Shyam Ramakrishnan, Brighton, MA (US)

Correspondence Address: BANNER & WITCOFF, 1001 G STREET N W, SUITE 1100, WASHINGTON, DC, 20001, US

(21) Appl. No.: 10/149,256

(22) PCT Filed: Dec. 1, 2000
(86) PCT No.: PCT/EP00/12110

(30) Foreign Application Priority Data
Dec. 8, 1999 (US) 60169615
Oct. 11, 2000 (US) 60239106

(51) Int. Cl. C12Q 1/68; C07H 21/04; C12N 9/18; C12P 21/02; C12N 5/06

(52) U.S. Cl. 435/6; 435/197; 435/69.1; 435/320.1; 435/325; 536/23.2

(57) ABSTRACT

Reagents which regulate human mitochondrial deformylase and reagents which bind to human mitochondrial deformylase gene products can be used to regulate cell proliferation, particularly in diseases such as cancer and other forms of neoplasia.

### TECHNICAL FIELD OF THE INVENTION

The invention relates to the area of regulation of cell proliferation. More particularly, the invention relates to the regulation of human mitochondrial deformylase activity to increase or decrease cell proliferation.

### BACKGROUND OF THE INVENTION

Traditional methods of treating diseases characterized by aberrant cell proliferation, such as cancer, involve agents which inhibit rapid cell division. Rapidly dividing cells require functional mitochondria to support cell division and maintenance. Thus, agents which inhibit mitochondrial protein synthesis have been used to treat cancer. Such methods also affect non-cancer cells which divide rapidly, however, which can result in unpleasant and potentially serious side effects. Thus, there is a need in the art for methods of regulating cell proliferation for the treatment of cancer and other proliferation-related diseases which will avoid or minimize the side effects associated with traditional treatment methods.

### SUMMARY OF THE INVENTION

The invention is based on the identification of a human mitochondrial deformylase. Based on in this identification the invention provides: 1) isolated mitochondrial deformylase protein or a biological active derivative thereof, 2) isolated nucleic acid molecules that encode the mitochondrial deformylase, 3) methods of isolating allelic variants of the mitochondrial deformylase protein and gene and 4) methods of identifying cells and tissues that express the mitochondrial deformylase gene/protein.

It is another object of the invention to provide reagents and methods of regulating cell proliferation and treating disease characterized by aberrant cell proliferation. These and other objects of the invention are provided by one or more of the embodiments described below.

One embodiment of the invention is a method of screening for agents which decrease proliferation of a cell. A test compound is contacted with a polypeptide. The polypeptide comprises an amino acid sequence selected from the group consisting of amino acid sequences which are at least about 50% identical to the amino acid sequence shown in SEQ ID NO:2, the amino acid sequence shown in SEQ ID NO:2, amino acid sequences which are at least about 50% identical to the amino acid sequence shown in SEQ ID NO:4, the amino acid sequence shown in SEQ ID NO:4, amino acid sequences which are at least about 50% identical to the amino acid sequence shown in SEQ ID NO:6, and the amino acid sequence shown in SEQ ID NO:6. Binding of the test compound to the polypeptide is detected. A test compound which binds to the target polypeptide is identified as a potential agent for decreasing proliferation of the cell.

Another embodiment of the invention is a method of screening for agents which regulate proliferation of a cell. A test compound is contacted with a polypeptide comprising an amino acid sequence selected from the group consisting of SEQ ID NOS:2, 4 and 6. A deformylase activity of the polypeptide is detected. A test compound which decreases the deformylase activity of the polypeptide relative to deformylase activity in the absence of the test compound is identified as a potential agent for decreasing proliferation of a cell. A test compound which increases the deformylase activity of the polypeptide relative to deformylase activity in the absence of the test compound is identified as a potential agent for increasing proliferation of a cell.

Another embodiment of the invention is a method of screening for agents which decrease proliferation of a cell. A test compound is contacted with a product of a polynucleotide comprising a nucleotide sequence selected from the group consisting of nucleotide sequences which are at least about 50% identical to the nucleotide sequence shown in SEQ ID NO:1, the nucleotide sequence shown in SEQ ID NO:1, nucleotide sequences which are at least about 50% identical to the nucleotide sequence shown in SEQ ID NO:3, the nucleotide sequence shown in SEQ ID NO:3, nucleotide sequences which are at least about 50% identical to the nucleotide sequence shown in SEQ ID NO:5, and the nucleotide sequence shown in SEQ ID NO:5. Binding of the test compound to the product is detected. A test compound which binds to the product is identified as a potential agent for decreasing proliferation of the cell.

Even another embodiment of the invention is a method of reducing proliferation of a cell. A cell is contacted with a reagent which specifically binds to a product encoded by a polynucleotide. The polynucleotide comprises a nucleotide sequence selected from the group consisting of nucleotide sequences which are at least about 50% identical to the nucleotide sequence shown in SEQ ID NO:1, the nucleotide sequence shown in SEQ ID NO:1, nucleotide sequences which are at least about 50% identical to the nucleotide sequence shown in SEQ ID NO:3, the nucleotide sequence shown in SEQ ID NO:3, nucleotide sequences which are at least about 50% identical to the nucleotide sequence shown in SEQ ID NO:5, and the nucleotide sequence shown in SEQ ID NO:5. Proliferation of the cell is thereby decreased.

The invention thus provides reagents and methods for regulating cell proliferation, particularly proliferation of neoplastic cells.

### BRIEF DESCRIPTION OF THE DRAWINGS

FIG. 1. BLASTP alignment of the partial human human mitochondrial deformylase protein (SEQ ID NO:4) with the putative full-length human mitochondrial deformylase protein as predicted by the Genescan algorithm (SEQ ID:6).

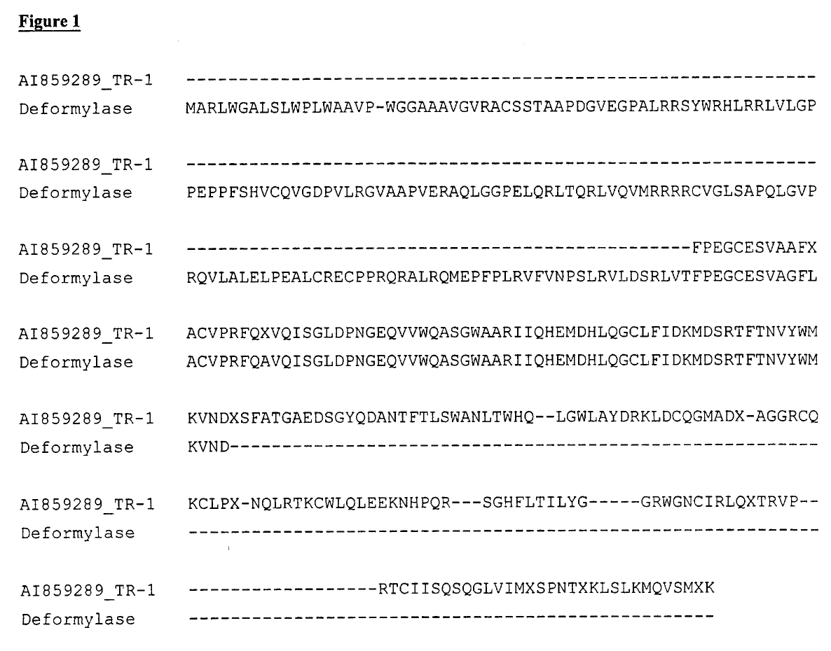

FIG. 2. Output of the Genescan algorithm: Analysis of human genomic sequence (AC026474) and prediction of the gene structure of the human mitochondrial deformylase gene

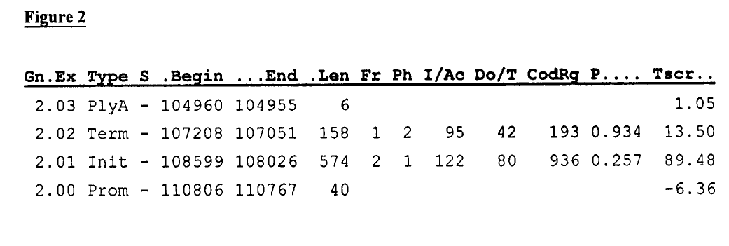

### DETAILED DESCRIPTION OF THE INVENTION

All technical terms used herein have the same meaning as is commonly used by those skilled in the art to which the present invention belongs.

“Nucleotide sequence” as used herein refers to an oligonucleotide, nucleotide or polynucleotide sequence, and fragments or portions thereof, and to DNA or RNA of genomic or synthetic origin which may be double-stranded or single-stranded whether representing the sense or the antisense strand.

The term “under stringent conditions” as used herein includes, but is not limited to, the condition of 65° C. for 10 to 2.0 hours in a solution containing 6×SSC, 1% sodium lauryl sulfate, 100 μg/ml salmon sperm DNA and 5×Denhardt's solution.

Similarly, an amino acid sequence as used herein refers to peptide or protein sequences or portions thereof. “Amino acid sequences wherein a substitution, deletion, addition, or transposition of one to several amino acid residue (s) is made” refers to modifications of the amino acid sequence that do not abolish the biological activity of the protein or protein fragment. Substitutions of the amino acid sequence may be “conservative”, when substituted amino acids have similar structural or chemical properties, e.g., replacement of leucine with isoleucine, or, more rarely, “non-conceservative”, when the structural or chemical properties of the exchanged amino acids are different, e.g. replacement of glycine with tryptophan.

“Functional equivalent” in connection with nucleic acids are derivatives of these nucleic acids which have the same function, that is it codes for the same or a quite similar protein. “Functional equivalent” in connection with proteins are derivatives or fragments of that protein which still have the same or quite the same biological function. An enzyme for example reacts with the same substrate and a receptor binds the same ligand. A derivative of a protein is a protein which can have additions, deletions, transitions and/or substitutions of amino acids compared to the disclosed sequence.

It is a discovery of the present invention that regulators of human mitochondrial deformylase can be used to treat diseases characterized by aberrant cell proliferation, such as cancer. Mitochondrial deformylase cleaves the formyl group from nascent formyl-methionine peptides in the following reaction: N-formyl-Met-peptide+H₂O® formate+N-met-peptide (see Meinnel et al., Biochimie 75, 1061-75, 1993, for a discussion of the related bacterial enzyme, peptide deformylase). Inhibition of mitochondrial deformylase according to the invention can affect protein production in rapidly dividing cells without significantly affecting protein production in normal cells. This decrease in mitochondrial protein production in rapidly dividing cancer cells leads to a decrease in proliferation of the cancer cells. Alternatively, according to the invention, cell proliferation can be increased by increasing mitochondrial deformylase activity.

### Mitochondrial Deformylase Polypeptides

Mitochondrial deformylase polypeptides according to the invention comprise an amino acid sequence as shown in SEQ ID NO:2, 4 or 6 or amino acid sequences wherein a substitution, deletion, addition or transposition of one to several amino acid residue(s) is made in SEQ ID NO:2, 4 or 6 or a biologically active variant of an amino acid sequence shown in SEQ ID NO:2, 4 or 6, as defined below. The undefined amino acids in SEQ ID NOS:2 and 4 represent the positions of stop codons introduced into SEQ ID NOS:1 and 3 (which encode SEQ ID NOS:2 and 4, respectively) by sequencing errors. A mitochondrial deformylase polypeptide of the invention can be a portion of a mitochondrial deformylase molecule, a full-length mitochondrial deformylase molecule, or a fusion protein comprising all or a portion of a mitochondrial deformylase molecule. Preferably, a mitochondrial deformylase polypeptide comprises the HEXXH motif (SEQ ID NO:7), which is typical of the active site of zinc-dependent metallopeptidases, including mitochondrial deformylase. Most preferably, a mitochondrial deformylase polypeptide has a deformylase activity. Deformylase activity is the removal of the formyl group from nascent formyl-methionine-peptides and is preferably measured as described in Adams, J. Mol. Biol. 33, 571-89, as modified by Meinnel & Blanquet, J. Bacteriol. 177, 1883-87 (1995), or WO 99/57097 (see also Examples 2 and 5, below).

### Biologically Active Variants

Mitochondrial deformylase variants which retain a mitochondrial deformylase activity, i.e., are biologically active, also are mitochondrial deformylase polypeptides. Preferably, naturally or non-naturally occurring mitochondrial deformylase variants have amino acid sequences which are at least about 50, preferably about 75, 90, 96, or 98% identical to amino acid sequences shown in SEQ ID NOS:2, 4 or 6. Percent identity between a putative mitochondrial deformylase variant and the amino acid sequence of SEQ ID NO:2, 4 or 6 can be determined, for example, using the Blast2 alignment program.

Variations in percent identity can be due, for example, to amino acid substitutions, insertions, or deletions. Amino acid substitutions are defined as one for one amino acid replacements. They are conservative in nature when the substituted amino acid has similar structural and/or chemical properties. Examples of conservative replacements are substitution of a leucine with an isoleucine or valine, an aspartate with a glutamate, or a threonine with a serine.

Amino acid insertions or deletions are changes to or within an amino acid sequence. They typically fall in the range of about 1 to 5 amino acids. Guidance in determining which amino acid residues can be substituted, inserted, or deleted without abolishing biological or immunological activity can be found using computer programs well known in the art, such as DNASTAR software. Whether an amino acid change results in a biologically active mitochondrial deformylase polypeptide can readily be determined by assaying for mitochondrial deformylase activity, as described, for example, in Adams, J. Mol. Biol. 33, 571-89, as modified by Meinnel & Blanquet, J. Bacteriol. 177, 1883-87 (1995), or WO 99/57097 (see also Examples 2 and 5, below).

### Fusion Proteins

Fusion proteins can comprise at least 6, 10, 20, 50, 75, 100, 150, or 200 or more contiguous amino acids of SEQ ID NO:2 or at least 6, 10, 20, 50, 75, 100, or 150 or more contiguous amino acids of SEQ ID NO:4 or at least 6, 10, 20, 50, 75, 100, or 150 or more contiguous amino acids of SEQ ID NO:6. Fusion proteins are useful for generating antibodies against mitochondrial deformylase amino acid sequences and for use in various assay systems. For example, fusion proteins can be used to identify proteins which interact with portions of a mitochondrial deformylase polypeptide, including its active site. Physical methods, such as protein affinity chromatography, or library-based assays for protein-protein interactions, such as the yeast two-hybrid or phage display systems, can also be used for this purpose. Such methods are well known in the art and can also be used as drug screens.

A mitochondrial deformylase fusion protein comprises two protein segments fused together by means of a peptide bond. The first protein segment comprises at least 6, 10, 20, 50, 75, 100, 150, or 200 or more contiguous amino acids of SEQ ID NO:2 or at least 6, 10, 20, 50, 75, 100, or 150 or more contiguous amino acids of SEQ ID NO:4 or at least 6, 10, 20, 50, 75, 100, or 150 or more contiguous amino acids of SEQ ID NO:6. Preferably, a fusion protein comprises the active site of a mitochondrial deformylase molecule. Contiguous mitochondrial deformylase amino acids for use in a fusion protein can be selected from the amino acid sequences shown in SEQ ID NO:2, 4 or 6 or from a biologically active variant of that sequence, such as those described above. The first protein segment can also comprise full-length mitochondrial deformylase.

The second protein segment can be a full-length protein or a protein fragment or polypeptide. Proteins commonly used in fusion protein construction include β-galactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags are used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and herpes simplex virus (HSV) BP16 protein fusions. A fusion protein can also be engineered to contain a cleavage site located between the mitochondrial deformylase polypeptide-encoding sequence and the heterologous protein sequence, so that the mitochondrial deformylase polypeptide can be cleaved and purified away from the heterologous moiety.

A fusion protein can be synthesized chemically, as is known in the art. Preferably, a fusion protein is produced by covalently linking two protein segments or by standard procedures in the art of molecular biology. Recombinant DNA methods can be used to prepare fusion proteins, for example, by making a DNA construct which comprises coding sequences selected from SEQ ID NO:1, 3 or 5 (encoding SEQ ID NOS:2, 4 and 6, respectively) in proper reading frame with nucleotides encoding the second protein segment and expressing the DNA construct in a host cell, as is known in the art. Many kits for constructing fusion proteins are available from companies such as Promega Corporation (Madison, Wis.), Stratagene (La Jolla, Calif.), Clontech (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

### Identification of Species Homologs

Species homologs of human mitochondrial deformylase can be obtained using mitochondrial deformylase polynucleotides (described below) to make suitable probes or primers to screening cDNA expression libraries from other species, such as mice, monkeys, or yeast, identifying cDNAs which encode homologs of mitochondrial deformylase, and expressing the cDNAs as is known in the art.

### Mitochondrial Deformylase Polynucleotides

A mitochondrial deformylase polynucleotide comprises a coding sequence for at least a portion of a mitochondrial deformylase polypeptide. For example, nucleotide sequences of mitochondrial deformylase polynucleotides which encode the mitochondrial deformylase polypeptides shown in SEQ ID NOS:2, 4 and 6 are shown in SEQ ID NOS:1 (AL045195), 3 (AI859289) and 5, respectively. Further cDNA sequences that were identified as partial mitochondrial deformylase genes are accessible through the EMBL database with the accession numbers: AI859289, AI765656, AW452869, AA831012, AW131443, AI394056, AW305385, AI363505, AI990860, AI651000, AA648991, AA714004, AA516472, AI991677, AI823498, AA112466, AI684882, AW001656, AA928208, A1623289, AI636515, AA746387, R42230 und AA715557.

Degenerate nucleotide sequences encoding amino acid sequences of human mitochondrial deformylase polypeptides, as well as homologous nucleotide sequences which are at least about 50, preferably about 75, 90, 96, or 98% identical to the nucleotide sequences shown in SEQ ID NOS:1, 3 or 5, are also mitochondrial deformylase polynucleotides. Percent sequence identity between the sequences of two polynucleotides is determined using computer programs such as ALIGN which employ the FASTA algorithm, using an affine gap search with a gap open penalty of −12 and a gap extension penalty of −2. Complementary DNA (cDNA) molecules, species homologs, and variants of human mitochondrial deformylase polynucleotides which encode biologically active mitochondrial deformylase polypeptides also are mitochondrial deformylase polynucleotides.

### Identification of Variants and Homologs of Mitochondrial Deformylase Polynucleotides

Variants and homologs of polynucleotides comprising a nucleotide sequence shown in SEQ ID NOS:1, 3 or 5 also are mitochondrial deformylase polypeptides. Typically, homologous mitochondrial deformylase polynucleotide sequences can be identified by hybridization of candidate polynucleotides to known mitochondrial deformylase polynucleotides under stringent conditions, as is known in the art. For example, using the following wash conditions—2×SSC (0.3 M NaCl, 0.03 M sodium citrate, pH 7.0), 0.1% SDS, room temperature twice, 30 minutes each; then 2×SSC, 0.1% SDS, 50° C. once, 30 minutes; then 2×SSC, room temperature twice, 10 minutes each—homologous sequences can be identified which contain at most about 25-30% basepair mismatches. More preferably, homologous nucleic acid strands contain 15-25% basepair mismatches, even more preferably 5-15% basepair mismatches.

Species homologs of the mitochondrial deformylase polynucleotides disclosed herein also can be identified by making suitable probes or primers and screening cDNA expression libraries from other species, such as mice, monkeys, or yeast. Human variants of mitochondrial deformylase polynucleotides can be identified by screening human cDNA expression libraries. It is well known that the Tₘ of a double-stranded DNA decreases by 1-1.5° C. with every 1% decrease in homology (Bonner et al., J. Mol. Biol. 81, 123 (1973). Variants of human mitochondrial deformylase polynucleotides or mitochondrial deformylase polynucleotides of other species can therefore be identified, for example, by hybridizing a putative homologous mitochondrial deformylase polynucleotide with a polynucleotide having the nucleotide sequence of SEQ ID NO:1, 3 or 5 to form a test hybrids, comparing the melting temperature of the test hybrid with the melting temperature of a hybrid comprising a polynucleotide having SEQ ID NO:1, 3 or 5 and a polynucleotide which is perfectly complementary to SEQ ID NO:1, 3 or 5, and calculating the number or percent of basepair mismatches within the test hybrid.

Nucleotide sequences which hybridize to the nucleotide sequences shown in SEQ ID NOS:1, 3 or 5 or their complements following stringent hybridization and/or wash conditions are also mitochondrial deformylase polynucleotides. Stringent wash conditions are well known and understood in the art and are disclosed, for example, in Sambrook et al., MOLECULAR CLONING: A LABORATORY MANUAL, 2d ed., 1989, at pages 9.50-9.51.

Typically, for stringent hybridization conditions a combination of temperature and salt concentration should be chosen that is approximately 12-20° C. below the calculated Tₘ of the hybrid under study. The Tₘ of a hybrid between the mitochondrial deformylase polynucleotide sequence shown in SEQ ID NO:1, 3 or 5 and a polynucleotide sequence which is at least about 50, preferably about 75, 90, 96, or 98% identical to SEQ ID NO:1, 3 or 5 can be calculated, for example, using the equation of Bolton and McCarthy, Proc. Natl. Acad. Sci U.S.A. 48, 1390 (1962):

Tₘ=81.5° C.−16.6(log₁₀[Na⁺])+0.41(%G+C)−0.63(%formamide)−600/l),

where l=the length of the hybrid in basepairs.

Stringent wash conditions include, for example, 4×SSC at 65° C., or 50% formamide, 4×SSC at 42° C., or 0.5×SSC, 0.1% SDS at 65° C. Highly stringent wash conditions include, for example, 0.2×SSC at 65° C.

### Preparation of Mitochondrial Deformylase Polynucleotides

A naturally occurring mitochondrial deformylase polynucleotide can be isolated free of other cellular components such as membrane components, proteins, and lipids. Polynucleotides can be made by a cell and isolated using standard nucleic acid purification techniques, or synthesized using an amplification technique, such as PCR, or by using an automatic synthesizer. Methods for isolating polynucleotides are routine and are known in the art. Any such technique for obtaining a polynucleotide can be used to obtain isolated mitochondrial deformylase polynucleotides. For example, restriction enzymes and probes can be used to isolate polynucleotide fragments which comprise nucleotide sequences encoding a mitochondrial deformylase polypeptide. Isolated polynucleotides are in preparations which are free or at least 70, 80, or 90% free of other molecules.

Mitochondrial deformylase cDNA molecules can be made with standard molecular biology techniques, using mitochondrial deformylase mRNA as a template. Mitochondrial deformylase cDNA molecules can thereafter be replicated using molecular biology techniques known in the art and disclosed in manuals such as Sambrook et al. (1989). An amplification technique, such as the polymerase chain reaction (PCR), can be used to obtain additional copies of subgenomic polynucleotides of the invention, using either human genomic DNA or cDNA as a template.

Alternatively, synthetic chemistry techniques can be used to synthesize mitochondrial deformylase polynucleotides. The degeneracy of the genetic code allows alternate nucleotide sequences to be synthesized which will encode a mitochondrial deformylase polypeptide having, for example, the amino acid sequence shown in SEQ ID NO:2, 4 or 6 or a biologically active variant of those sequences.

### Obtaining Full-Length Mitochondrial Deformylase Polynucleotides

The partial sequences of SEQ ID NOS:1 and 3 and the putative full-length sequence of SEQ ID NO:5 can be used to verify experimentally the corresponding fill length gene from which they are derived. The partial sequences can be nick-translated or end-labeled with ³²P using polynucleotide kinase using labeling methods known to those with skill in the art (BASIC METHODS IN MOLECULAR BIOLOGY, Davis et al., eds., Elsevier Press, N.Y., 1986). A lambda library prepared from human tissue can be directly screened with the labeled sequences of interest or the library can be converted en masse to pBluescript (Stratagene Cloning Systems, La Jolla, Calif. 92037) to facilitate bacterial colony screening (see Sambrook et al., MOLECULAR CLONING: A LABORATORY MANUAL, Cold Spring Harbor Laboratory Press (1989, pg. 1.20).

Both methods are well known in the art. Briefly, filters with bacterial colonies containing the library in pBluescript or bacterial lawns containing lambda plaques are denatured, and the DNA is fixed to the filters. The filters are hybridized with the labeled probe using hybridization conditions described by Davis et al., 1986. The partial sequences, cloned into lambda or pBluescript, can be used as positive controls to assess background binding and to adjust the hybridization and washing stringencies necessary for accurate clone identification. The resulting auto-radiograms are compared to duplicate plates of colonies or plaques; each exposed spot corresponds to a positive colony or plaque. The colonies or plaques are selected, expanded and the DNA is isolated from the colonies for further analysis and sequencing.

Positive cDNA clones are analyzed to determine the amount of additional sequence they contain using PCR with one primer from the partial sequence and the other primer from the vector. Clones with a larger vector-insert PCR product than the original partial sequence are analyzed by restriction digestion and DNA sequencing to determine whether they contain an insert of the same size or similar as the mRNA size determined from Northern blot Analysis.

Once one or more overlapping cDNA clones are identified, the complete sequence of the clones can be determined, for example after exonuclease III digestion (McCombie et al., Methods 3, 33-40, 1991). A series of deletion clones are generated, each of which is sequenced. The resulting overlapping sequences are assembled into a single contiguous sequence of high redundancy (usually three to five overlapping sequences at each nucleotide position), resulting in a highly accurate final sequence.

Various PCR-based methods can be used to extend the nucleic acid sequences encoding the disclosed portions of human mitochondrial deformylase to detect upstream sequences such as promoters and regulatory elements. For example, restriction-site PCR uses universal primers to retrieve unknown sequence adjacent to a known locus (Sarkar, PCR Methods Applic. 2, 318-322, 1993). In particular, genomic DNA is first amplified in the presence of primer to a linker sequence and a primer specific to the known region. The amplified sequences are then subjected to a second round of PCR with the same linker primer and another specific primer internal to the first one. Products of each round of PCR are transcribed with an appropriate RNA polymerase and sequenced using reverse transcriptase.

Inverse PCR also can be used to amplify or extend sequences using divergent primers based on a known region (Triglia et al., Nucleic Acids Res. 16, 8186, 1988). Primers can be designed using commercially available software, such as OLIGO 4.06 Primer Analysis software (National Biosciences Inc., Plymouth, Minn.), to be 22-30 nucleotides in length, to have a GC content of 50% or more, and to anneal to the target sequence at temperatures about 68°-72° C. The method uses several restriction enzymes to generate a suitable fragment in the known region of a gene. The fragment is then circularized by intramolecular ligation and used as a PCR template.

Another method which may be used is capture PCR which involves PCR amplification of DNA fragments adjacent to a known sequence in human and yeast artificial chromosome DNA (Lagerstrom et al., PCR Methods Applic. 1, 111-119, 1991). In this method, multiple restriction enzyme digestions and ligations may also be used to place an engineered double-stranded sequence into an unknown fragment of the DNA molecule before performing PCR.

Another method which may be used to retrieve unknown sequences is that of Parker et al., Nucleic Acids Res. 19, 3055-3060 (1991). Additionally, PCR, nested primers, and PROMOTERFINDER libraries (Clontech, Palo Alto, Calif.) can be used to walk genomic DNA (Clontech, Palo Alto, Calif.). This process avoids the need to screen libraries and is useful in finding intron/exon junctions.

When screening for full-length cDNAs, it is preferable to use libraries that have been size-selected to include larger cDNAs. Also, random-primed libraries are preferable, in that they will contain more sequences which contain the 5′ regions of genes. Use of a randomly primed library may be especially preferable for situations in which an oligo d(T) library does not yield a full-length cDNA. Genomic libraries may be useful for extension of sequence into 5′ non-transcribed regulatory regions. Commercially available capillary electrophoresis systems can be used to analyze the size or confirm the nucleotide sequence of sequencing or PCR products. In particular, capillary sequencing may employ flowable polymers for electrophoretic separation, four different fluorescent dyes (one for each nucleotide) which are laser activated, and detection of the emitted wavelengths by a charge coupled device camera. Output/light intensity can be converted to electrical signal using appropriate software (e.g. GENOTYPER and Sequence NAVIGATOR, Perkin Elmer), and the entire process from loading of samples to computer analysis and electronic data display can be computer controlled. Capillary electrophoresis is especially preferable for the sequencing of small pieces of DNA which might be present in limited amounts in a particular sample.

### Obtaining Mitochondrial Deformylase Polypeptides

Mitochondrial deformylase polypeptides can be obtained, for example, by purification from human cells, by expression of mitochondrial deformylase poly-nucleotides, or by direct chemical synthesis.

### Protein Purification

Mitochondrial deformylase polypeptides can be purified from human cells, preferably using the method of Meinnel & Blanquet, J. Bacteriol. 175, 7737-40 (1993), as modified by Meinnel & Blanquet (1995). A purified mitochondrial deformylase polypeptide is separated from other compounds which normally associate with the mitochondrial deformylase polypeptide in the cell, such as certain proteins, carbohydrates, or lipids. A preparation of purified mitochondrial deformylase polypeptides is at least 80% pure; preferably, the preparations are 90%, 95%, or 99% pure. Purity of the preparations can be assessed by any means known in the art, such as SDS-polyacrylamide gel electrophoresis.

### Expression of Mitochondrial Deformylase Polynucleotides

To express a mitochondrial deformylase polypeptide, a mitochondrial deformylase polynucleotide can be inserted into an appropriate expression vector, i.e., a vector which contains the necessary elements for the transcription and translation of the inserted coding sequence. Methods which are well known to those skilled in the art may be used to construct expression vectors containing sequences encoding mitochondrial deformylase polypeptides and appropriate transcriptional and translational control elements. These methods include in vitro recombinant DNA techniques, synthetic techniques, and in vivo genetic recombination. Such techniques are described, for example, in Sambrook et al. (1989) and Ausubel et al., CURRENT PROTOCOLS IN MOLECULAR BIOLOGY, John Wiley & Sons, New York, N.Y, 1989.

A variety of expression vector/host systems may be utilized to contain and express sequences encoding a mitochondrial deformylase polypeptide. These include, but are not limited to: microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems.

The control elements or regulatory sequences are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the BLUESCRIPT phagemid (Stratagene, LaJolla, Calif.) or pSPORT1 plasmid (Life Technologies) and the like may be used. The baculovirus polyhedrin promoter may be used in insect cells. Promoters or enhancers derived from the genomes of plant cells (e.g., heat shock, RUBISCO; and storage protein genes) or from plant viruses (e.g., viral promoters or leader sequences) may be cloned into the vector. In mammalian cell systems, promoters from mammalian genes or from mammalian viruses are preferable. If it is necessary to generate a cell line that contains multiple copies of a nucleotide sequence encoding a mitochondrial deformylase polypeptide, vectors based on SV40 or EBV may be used with an appropriate selectable marker.

In bacterial systems, a number of expression vectors may be selected depending upon the use intended for the mitochondrial deformylase polypeptide. For example, when a large quantity of a mitochondrial deformylase polypeptide is needed for the induction of antibodies, vectors which direct high level expression of fusion proteins that are readily purified may be used. Such vectors include, but are not limited to: multifunctional E. coli cloning and expression vectors such as BLUESCRIPT (Stratagene), in which the sequence encoding the mitochondrial deformylase polypeptide may be ligated into the vector in frame with sequences for the amino-terminal Met and the subsequent 7 residues of β-galactosidase so that a hybrid protein is produced, pIN vectors (Van Heeke & Schuster, J. Biol. Chem. 264, 5503-5509, 1989. pGEX vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In the yeast Saccharomyces cerevisiae, a number of vectors containing constitutive or inducible promoters such as alpha factor, alcohol oxidase, and PGH may be used. For reviews, see Ausubel et al. (1989) and Grant et al., Methods Enzymol. 153, 516-544, 1987.

In cases where plant expression vectors are used, the expression of sequences encoding mitochondrial deformylase polypeptides can be driven by any of a number of promoters. For example, viral promoters such as the 35S and 19S promoters of CaMV may be used alone or in combination with the omega leader sequence from TMV (Takamatsu EMBO J. 6, 307-311, 1987). Alternatively, plant promoters such as the small subunit of RUBISCO or heat shock promoters may be used (Coruzzi et al., EMBO J. 3, 1671-1680, 1984; Broglie et al., Science 224, 838-843, 1984; and Winter et al., Results Probl. Cell Differ. 17, 85-105, 1991). These constructs can be introduced into plant cells by direct DNA transformation or pathogen-mediated transfection. Such techniques are described in a number of generally available reviews (see, for example, Hobbs or Murry, in McGRAW HILL YEARBOOK OF SCIENCE AND TECHNOLOGY, McGraw Hill, New York, N.Y., pp. 191-196, 1992).

An insect system may also be used to express a mitochondrial deformylase polypeptide. For example, in one such system Autographa californica nuclear polyhedrosis virus (AcNPV) is used as a vector to express foreign genes in Spodoptera frugiperda cells or in Trichoplusia larvae. Sequences encoding mitochondrial deformylase polypeptides can be cloned into a non-essential region of the virus, such as the polyhedrin gene, and placed under control of the polyhedrin promoter.

Successful insertion of mitochondrial deformylase polypeptides will render the polyhedrin gene inactive and produce recombinant virus lacking coat protein. The recombinant viruses may then be used to infect, for example, S. frugiperda cells or Trichoplusia larvae in which mitochondrial deformylase polypeptides may be expressed (Engelhard et al., (1994) Proc. Nat. Acad. Sci. 91, 3224-3227, 1994).

A number of viral-based expression systems can be utilized in mammalian host cells. In cases where an adenovirus is used as an expression vector, sequences encoding mitochondrial deformylase polypeptides can be ligated into an adenovirus transcription/translation complex consisting of the late promoter and tripartite leader sequence. Insertion in a non-essential E1 or E3 region of the viral genome may be used to obtain a viable virus which is capable of expressing mitochondrial deformylase polypeptide in infected host cells (Logan & Shenk, Proc. Natl. Acad. Sci. 81, 3655-3659, 1984). In addition, transcription enhancers, such as the Rous sarcoma virus (RSV) enhancer, may be used to increase expression in mammalian host cells.

Human artificial chromosomes (HACs) may also be employed to deliver larger fragments of DNA than can be contained and expressed in a plasmid. HACs of 6 to 10M are constructed and delivered via conventional delivery methods (liposomes, polycationic amino polymers, or vesicles) for therapeutic purposes.

Specific initiation signals may also be used to achieve more efficient translation of sequences encoding mitochondrial deformylase polypeptides. Such signals include the ATG initiation codon and adjacent sequences. In cases where sequences encoding a mitochondrial deformylase polypeptide, its initiation codon, and upstream sequences are inserted into the appropriate expression vector, no additional transcriptional or translational control signals may be needed. However, in cases where only coding sequence, or a fragment thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used (see Scharf et al., Results Probl. Cell Differ. 20, 125-162, 1994).

A host cell strain can be chosen for its ability to modulate the expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding and/or function. Different host cells which have specific cellular machinery and characteristic mechanisms for post-translational activities (e.g., CHO, HeLa, MDCK, HEK293, and W138), are available from the American Type Culture Collection (ATCC; 10801 University Boulevard, Manassas, Va. 20110-2209) and may be chosen to ensure the correct modification and processing of the foreign protein.

Stable expression is preferred for long-term, high-yield production of recombinant proteins. For example, cell lines which stably express mitochondrial deformylase polypeptides may be transformed using expression vectors which may contain viral origins of replication and/or endogenous expression elements and a selectable marker gene on the same or on a separate vector. Following the introduction of the vector, cells may be allowed to grow for 1-2 days in an enriched medium before they are switched to a selective medium. The purpose of the selectable marker is to confer resistance to selection, and its presence allows growth and recovery of cells which successfully express the introduced sequences. Resistant clones of stably transformed cells may be proliferated using tissue culture techniques appropriate to the cell type.

Any number of selection systems may be used to recover transformed cell lines. These include, but are not limited to, the herpes simplex virus thymidine kinase (Wigler et al., Cell 11, 223-32, 1977) and adenine phosphoribosyltransferase (Lowy et al., Cell 22, 817-23, 1980) genes which can be employed in tk⁻ or aprt⁻ cells, respectively. Also, antimetabolite, antibiotic, or herbicide resistance can be used as the basis for selection; for example, dhfr confers resistance to methotrexate (Wigler et al., Proc. Natl. Acad. Sci. 77, 3567-70, 1980); npt, confers resistance to the aminoglycosides, neomycin and G-418 (Colbere-Garapin et al., J. Mol. Biol. 150, 1-14, 1981), and als or pat confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman & Mulligan, Proc. Natl. Acad. Sci. 85, 8047-51, 1988). Visible markers such as anthocyanins, β-glucuronidase and its substrate GUS, and luciferase and its substrate luciferin, can be used to identify transformants and to quantify the amount of transient or stable protein expression attributable to a specific vector system (Rhodes et al., Methods Mol. Biol. 55, 121-131, 1995).

Although the presence of marker gene expression suggests that the mitochondrial deformylase polynucleotide is also present, its presence and expression may need to be confirmed. For example, if a sequence encoding a mitochondrial deformylase polypeptide is inserted within a marker gene sequence, transformed cells containing sequences which encode a mitochondrial deformylase polypeptide can be identified by the absence of marker gene function. Alternatively, a marker gene can be placed in tandem with a sequence encoding a mitochondrial deformylase polypeptide under the control of a single promoter. Expression of the marker gene in response to induction or selection usually indicates expression of the mitochondrial deformylase polynucleotide.

Alternatively, host cells which contain a mitochondrial deformylase polynucleotide and which express a mitochondrial deformylase polypeptide can be identified by a variety of procedures known to those of skill in the art. These procedures include, but are not limited to, DNA-DNA or DNA-RNA hybridizations and protein bioassay or immunoassay techniques which include membrane, solution, or chip-based technologies for the detection and/or quantification of nucleic acid or protein.

The presence of a polynucleotide sequence encoding a mitochondrial deformylase polypeptide can be detected by DNA-DNA or DNA-RNA hybridization or amplification using probes or fragments or fragments of polynucleotides encoding a mitochondrial deformylase polypeptide. Nucleic acid amplification-based assays involve the use of oligonucleotides selected from sequences encoding a mitochondrial deformylase polypeptide to detect transformants which contain a mitochondrial deformylase polynucleotide.

A variety of protocols for detecting and measuring the expression of a mitochondrial deformylase polypeptide, using either polyclonal or monoclonal antibodies specific for the polypeptide are known in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on a mitochondrial deformylase polypeptide is preferred, but a competitive binding assay can be employed. These and other assays are described, among other places, in Hampton et al., SEROLOGICAL METHODS: A LABORATORY MANUAL, APS Press, St Paul, Minn., 1990) and Maddox et al., J. Exp. Med. 158,1211-1216,1983).

A wide variety of labels and conjugation techniques are known by those skilled in the art and may be used in various nucleic acid and amino acid assays. Means for producing labeled hybridization or PCR probes for detecting sequences related to polynucleotides encoding mitochondrial deformylase polypeptides include oligolabeling, nick translation, end-labeling or PCR amplification using a labeled nucleotide. Alternatively, the sequences encoding a mitochondrial deformylase polypeptide, or any fragments thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits Amersham Pharmacia Biotech, Promega, and US Biochemical. Suitable reporter molecules or labels, which may be used for ease of detection, include radionuclides, enzymes, fluorescent, chemiluminescent, or chromogenic agents as well as substrates, cofactors, inhibitors, magnetic particles, and the like.

Host cells transformed with nucleotide sequences encoding mitochondrial deformylase polypeptides may be cultured under conditions suitable for the expression and recovery of the protein from cell culture. The protein produced by a transformed cell may be secreted or contained intracellularly depending on the sequence and/or the vector used. As will be understood by those of skill in the art, expression vectors containing polynucleotides which encode mitochondrial deformylase polypeptides may be designed to contain signal sequences which direct secretion of mitochondrial deformylase polypeptides through a prokaryotic or eukaryotic cell membrane.

Other constructions may be used to join sequences encoding mitochondrial deformylase polypeptides to nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). The inclusion of cleavable linker sequences such as those specific for Factor XA or enterokinase (Invitrogen, San Diego, Calif.) between the purification domain and mitochondrial deformylase polypeptides may be used to facilitate purification. One such expression vector provides for expression of a filsion protein containing mitochondrial deformylase polypeptides and a nucleic acid encoding 6 histidine residues preceding a thioredoxin or an enterokinase cleavage site. The histidine residues facilitate purification on IMAC (immobilized metal ion affinity chromatography as described in Porath et al., Prot. Exp. Purif. 3, 263-281, 1992) while the enterokinase cleavage site provides a means for purifying mitochondrial deformylase polypeptides from the fusion protein. A discussion of vectors which contain fusion proteins is provided in Kroll et al., DNA Cell Biol. 12, 441-453, 1993).

### Chemical Synthesis

In another embodiment, sequences encoding a mitochondrial deformylase polypeptide may be synthesized, in whole or in part, using chemical methods well known in the art (see Caruthers et al., Nucl. Acids Res. Symp. Ser. 215-223, 1980; Horn et al. Nucl. Acids Res. Symp. Ser. 225-232, 1980). Alternatively, the mitochondrial deformylase polypeptide itself may be produced using chemical methods to synthesize the amino acid sequence of the mitochondrial deformylase polypeptide, or a fragment thereof. For example, peptide synthesis can be performed using various solid-phase techniques (Roberge et al., Science 269, 202-204, 1995) and automated synthesis may be achieved, for example, using the ABI 431A Peptide Synthesizer (Perkin Elmer).

In addition to recombinant production, fragments of mitochondrial deformylase polypeptides may be produced by direct peptide synthesis using solid-phase techniques (Merrifield, J. Am. Chem. Soc. 85, 2149-2154, 1963). Protein synthesis may be performed using manual techniques or by automation. Automated synthesis may be achieved, for example, using Applied Biosystems 431A Peptide Synthesizer (Perkin Elmer). Various fragments of mitochondrial deformylase polypeptides may be chemically synthesized separately and combined using chemical methods to produce the full length molecule.

The newly synthesized peptide may be substantially purified by preparative high performance liquid chromatography (e.g., Creighton, PROTEINS: STRUCTURES AND MOLECULAR PRINCIPLES, WH Freeman and Co., New York, N.Y., 1983). The composition of the synthetic peptides may be confirmed by amino acid analysis or sequencing (e.g., the Edman degradation procedure; see Creighton, supra). Additionally, the amino acid sequence of the polypeptide or any part thereof may be altered during direct synthesis and/or combined using chemical methods with sequences from other proteins, or any part thereof, to produce a variant polypeptide.

### Production of Altered Mitochondrial Deformylase Polypeptides

As will be understood by those of skill in the art, it may be advantageous to produce mitochondrial deformylase polypeptide-encoding nucleotide sequences possessing non-naturally occurring codons. For example, codons preferred by a particular prokaryotic or eukaryotic host can be selected to increase the rate of protein expression or to produce an RNA transcript having desirable properties, such as a half-life which is longer than that of a transcript generated from the naturally occurring sequence.

The nucleotide sequences disclosed herein can be engineered using methods generally known in the art in order to alter mitochondrial deformylase polypeptide-encoding sequences for a variety of reasons, including but not limited to, alterations which modify the cloning, processing, and/or expression of the gene product. DNA shuffling by random fragmentation and PCR reassembly of gene fragments and synthetic oligonucleotides may be used to engineer the nucleotide sequences. For example, site-directed mutagenesis may be used to insert new restriction sites, alter glycosylation patterns, change codon preference, produce splice variants, introduce mutations, and so forth.

### Antibodies

Any type of antibody known in the art can be generated to bind specifically to an epitope of a mitochondrial deformylase polypeptide. ,,Antibody” as used herein includes intact immunoglobulin molecules, as well as fragments thereof, such as Fa, F(ab′)₂, and Fv, which are capable of binding an epitope of a mitochondrial deformylase polypeptide. Typically, at least 6, 8, 10, or 12 contiguous amino acids are required to form an epitope. However, epitopes which involve non-contiguous amino acids may require more, e.g., at least 15, 25, or 50 amino acids.

An antibody which specifically binds to an epitope of a mitochondrial deformylase polypeptide can be used therapeutically, as well as in immunochemical assays, including but not limited to Western blots, ELISAs, radioimmunoassays, immunohistochemical assays, immunoprecipitations, or other immunochemical assays known in the art. Various immunoassays can be used to identify antibodies having the desired specificity. Numerous protocols for competitive binding or immunoradiometric assays are well known in the art. Such immunoassays typically involve the measurement of complex formation between an immunogen and an antibody which specifically binds to the immunogen.

Typically, an antibody which specifically binds to a mitochondrial deformylase polypeptide provides a detection signal at least 5-, 10-, or 20-fold higher than a detection signal provided with other proteins when used in such immunochemical assays. Preferably, antibodies which specifically bind to mitochondrial deformylase polypeptides do not detect other proteins in immunochemical assays and can immunoprecipitate a mitochondrial deformylase polypeptide from solution.

Mitochondrial deformylase polypeptides can be used to immunize a mammal, such as a mouse, rat, rabbit, guinea pig, monkey, or human, to produce polyclonal antibodies. If desired, the mitochondrial deformylase polypeptide can be conjugated to a carrier protein, such as bovine serum albumin, thyroglobulin, and keyhole limpet hemocyanin. Depending on the host species, various adjuvants can be used to increase the immunological response. Such adjuvants include, but are not limited to, Freund's adjuvant, mineral gels (e.g., aluminum hydroxide), and surface active substances (e.g. lysolecithin, pluronic polyols, polyanions, peptides, oil emulsions, keyhole limpet hemocyanin, and dinitrophenol). Among adjuvants used in humans, BCG (bacilli Calmette-Guerin) and Corynebacterium parvum are especially preferable.

Monoclonal antibodies which specifically bind to a mitochondrial deformylase polypeptide can be prepared using any technique which provides for the production of antibody molecules by continuous cell lines in culture. These techniques include, but are not limited to, the hybridoma technique, the human B-cell hybridoma technique, and the EBV-hybridoma technique (Kohler et al., Nature 256, 495-497, 1985; Kozbor et al., J. Immunol. Methods 81, 31-42, 1985; Cote et al., Proc. Natl. Acad. Sci. 80, 2026-2030, 1983; Cole et al., Mol. Cell Biol. 62, 109-120, 1984).

In addition, techniques developed for the production of ,,chimeric antibodies,” the splicing of mouse antibody genes to human antibody genes to obtain a molecule with appropriate antigen specificity and biological activity can be used (Morrison et al., Proc. Natl. Acad. Sci. 81, 6851-6855, 1984; Neuberger et al., Nature 312, 604-608, 1984; Takeda et al., Nature 314, 452-454, 1985). Monoclonal and other antibodies can also be ,,humanized” in order to prevent a patient from mounting an immune response against the antibody when it is used therapeutically. Such antibodies may be sufficiently similar in sequence to human antibodies to be used directly in therapy or may require alteration of a few key residues. Sequence differences between, for example, rodent antibodies and human sequences can be minimized by replacing residues which differ from those in the human sequences, for example, by site directed mutagenesis of individual residues, or by grating of entire complementarity determining regions. Alternatively, one can produce humanized antibodies using recombinant methods, as described in GB 2188638B. Antibodies which specifically bind to a mitochondrial deformylase polypeptide can contain antigen binding sites which are either partially or fully humanized, as disclosed in U.S. Pat. No. 5,565,332.

Alternatively, techniques described for the production of single chain antibodies may be adapted, using methods known in the art, to produce single chain antibodies which specifically bind to mitochondrial deformylase polypeptides. Antibodies with related specificity, but of distinct idiotypic composition, may be generated by chain shuffling from random combinatorial immunoglobin libraries (Burton, Proc. Natl. Acad. Sci. 88, 11120-23, 1991).

Single-chain antibodies can also be constructed using a DNA amplification method, such as the polymerase chain reaction (PCR), using hybridoma cDNA as a template (Thirion et al., 1996, Eur. J. Cancer Prev. 5, 507-11). Single-chain antibodies can be mono- or bispecific, and can be bivalent or tetravalent. Construction of tetravalent, bispecific single-chain antibodies is taught, for example, in Coloma & Morrison, 1997, Nat. Biotechnol. 15, 159-63. Construction of bivalent, bispecific single-chain antibodies is taught inter alia in Mallender & Voss, 1994, J. Biol. Chem. 269, 199-206.

A nucleotide sequence encoding a single-chain antibody can be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology. Verhaar et al., 1995, Int. J. Cancer 61, 497-501; Nicholls et al., 1993, J. Immunol. Meth. 165, 81-91.

Antibodies which specifically bind to mitochondrial deformylase polypeptides also can be produced by inducing in vivo production in the lymphocyte population or by screening immunoglobulin libraries or panels of highly specific binding reagents as disclosed in the literature (Orlandi et al., Proc. Natl. Acad. Sci. 86, 3833-3837, 1989; Winter et al., Nature 349, 293-299, 1991).

Other types of antibodies can be constructed and used therapeutically in methods of the invention. For example, chimeric antibodies can be constructed as disclosed in WO 93/03151. Binding proteins which are derived from immunoglobulins and which are multivalent and multispecific, such as the ,,diabodies” described in WO 94/13804, can also be prepared.

Antibodies of the invention can be purified by methods well known in the art. For example, antibodies can be affinity purified by passing the antibodies over a column to which a mitochondrial deformylase polypeptide is bound. The bound antibodies can then be eluted from the column, using a buffer with a high salt concentration.

### Antisense Oligonucleotides

Antisense oligonucleotides are nucleotide sequences which are complementary to a specific DNA or RNA sequence. Once introduced into a cell, the complementary nucleotides combine with natural sequences produced by the cell to form duplexes and block either transcription or translation. Preferably, an antisense oligonucleotide is at least 8 nucleotides in length, but can be at least 11, 12, 15, 20, 25, 30, 35, 40, 45, or 50 or more nucleotides long. Longer sequences can also be used. Antisense oligonucleotide molecules can be provided in a DNA construct and introduced into a cell as described above to decrease the level of mitochondrial deformylase gene products in the cell.

Antisense oligonucleotides can be deoxyribonucleotides, ribonucleotides, peptide nucleic acids (PNAs; described in U.S. Pat. No. 5,714,331), locked nuleic acids (LNAs; described in WO 99/14226), or a combination of them. Oligonucleotides can be synthesized manually or by an automated synthesizer, by covalently linking the 5′ end of one nucleotide with the 3′ end of another nucleotide with non-phosphodiester internucleotide linkages such alkylphosphonates, phosphorothioates, phosphorodithioates, alkylphosphonothioates, alkylphosphonates, phosphoramidates, phosphate esters, carbamates, acetamidate, carboxymethyl esters, carbonates, and phosphate triesters. See Brown, Meth. Mol. Biol. 20, 1-8, 1994; Sonveaux, Meth. Mol. Biol. 26, 1-72, 1994; Uhlmann et al., Chem. Rev. 90, 543-583, 1990.

Modifications of mitochondrial deformylase gene expression can be obtained by designing antisense oligonucleotides molecules which will form duplexes to the control, 5′ or regulatory regions of the mitochondrial deformylase encoding gene. Oligonucleotides derived from the transcription initiation site, e.g., between positions −10 and +10 from the start site, are preferred. Similarly, inhibition can be achieved using “triple helix” base-pairing methodology. Triple helix pairing is useful because it causes inhibition of the ability of the double helix to open sufficiently for the binding of polymerases, transcription factors, or chaperons. Recent therapeutic advances using triplex DNA have been described in the literature (Gee et al., in Huber & Carr, MOLECULAR AND IMMUNOLOGIC APPROACHES, Futura Publishing Co., Mt. Kisco, N.Y., 1994). The complementary sequence or antisense molecule may also be designed to block translation of mRNA by preventing the transcript from binding to ribosomes.

Precise complementarity is not required for successful duplex formation between an antisense oligonucleotide and the complementary sequence of a mitochondrial deformylase polynucleotide. Antisense molecules which comprise, for example, 2, 3, 4, or 5 or more stretches of contiguous nucleotides which are precisely complementary to a mitochondrial deformylase polynucleotide, each separated by a stretch of contiguous nucleotides which are not complementary to adjacent mitochondrial deformylase nucleotides, can provide targeting specificity for mitochondrial deformylase mRNA. Preferably, each stretch of contiguous nucleotides is at least 4, 5, 6, 7, or 8 or more nucleotides in length. Non-complementary intervening sequences are preferably 1, 2, 3, or 4 nucleotides in length. One skilled in the art can easily use the calculated melting point of an antisense-sense pair to determine the degree of mismatching which will be tolerated between a particular antisense oligonucleotide and a particular mitochondrial deformylase polynucleotide sequence.

Antisense oligonucleotides can be modified without affecting their ability to hybridize to a mitochondrial deformylase polynucleotide. These modifications can be internal or at one or both ends of the antisense molecule. For example, internucleoside phosphate linkages can be modified by adding cholesteryl or diamine moieties with varying numbers of carbon residues between the amino groups and terminal ribose. Modified bases and/or sugars, such as arabinose instead of ribose, or a 3′, 5′-substituted oligonucleotide in which the 3′ hydroxyl group or the 5′ phosphate group are substituted, can also be employed in a modified antisense oligonucleotide. These modified oligonucleotides can be prepared by methods well known in the art. See, e.g., Agrawal et al., Trends Biotechnol. 10, 152-158, 1992; Uhlmann et al., Chem. Rev. 90, 543-584, 1990; Uhlmann et al., Tetrahedron. Lett. 215, 3539-3542, 1987.

### Ribozymes

Ribozymes are RNA molecules with catalytic activity. See, e.g., Cech, Science 236, 1532-1539; 1987; Cech, Ann. Rev. Biochem. 59, 543-568; 1990, Cech, Curr. Opin. Struct. Biol. 2, 605-609; 1992, Couture & Stinchcomb, Trends Genet. 12, 510-515, 1996. Ribozymes can be used to inhibit gene function by cleaving an RNA sequence, as is known in the art (e.g., Haseloff et al., U.S. Pat. No. 5,641,673). The mechanism of ribozyme action involves sequence-specific hybridization of the ribozyme molecule to complementary target RNA, followed by endonucleolytic cleavage. Examples include engineered hammerhead motif ribozyme molecules that can specifically and efficiently catalyze endonucleolytic cleavage of specific nucleotide sequences.

The coding sequence of a mitochondrial deformylase polynucleotide can be used to generate ribozymes which will specifically bind to mRNA transcribed from the mitochondrial deformylase polynucleotide. Methods of designing and constructing ribozymes which can cleave other RNA molecules in trans in a highly sequence specific manner have been developed and described in the art (see Haseloff, J. et al. Nature 334, 585-591, 1988). For example, the cleavage activity of ribozymes can be targeted to specific RNAs by engineering a discrete “hybridization” region into the ribozyme. The hybridization region contains a sequence complementary to the target RNA and thus specifically hybridizes with the target (see, for example, Gerlach et al., EP 321,201).

Specific ribozyme cleavage sites within a mitochondrial deformylase RNA target are initially identified by scanning the target molecule for ribozyme cleavage sites which include the following sequences: GUA, GUU, and GUC. Once identified, short RNA sequences of between 15 and 20 ribonucleotides corresponding to the region of the target RNA containing the cleavage site may be evaluated for secondary structural features which may render the oligonucleotide inoperable. The suitability of candidate targets may also be evaluated by testing accessibility to hybridization with complementary oligonucleotides using ribonuclease protection assays. The nucleotide sequences shown in SEQ ID NOS: 1, 3 and 5 provide a source of suitable hybridization region sequences. Longer complementary sequences can be used to increase the affinity of the hybridization sequence for the target. The hybridizing and cleavage regions of the ribozyme can be integrally related; thus, upon hybridizing to the target RNA through the complementary regions, the catalytic region of the ribozyme can cleave the target.

Ribozymes can be introduced into cells as part of a DNA construct, as is known in the art and described above. Mechanical methods, such as microinjection, liposome-mediated transfection, electroporation, or calcium phosphate precipitation, can be used to introduce the ribozyme-containing DNA construct into cells in which it is desired to decrease mitochondrial deformylase expression, as described above.

Alternatively, if it is desired that the cells stably retain the DNA construct, it can be supplied on a plasmid and maintained as a separate element or integrated into the genome of the cells, as is known in the art. The DNA construct can include transcriptional regulatory elements, such as a promoter element, an enhancer or UAS element, and a transcriptional terminator signal, for controlling transcription of ribozymes in the cells.

As taught in Haseloff et al., U.S. Pat. No. 5,641,673, ribozymes can be engineered so that ribozyme expression will occur in response to factors which induce expression of a target gene. Ribozymes can also be engineered to provide an additional level of regulation, so that destruction of mRNA occurs only when both a ribozyme and a target gene are induced in the cells.

### Screening Methods

The invention provides methods for identifying modulators, i.e., candidate or test compounds which bind to mitochondrial deformylase polypeptides or polynucleotides and/or have a stimulatory or inhibitory effect on, for example, expression or activity of the mitochondrial deformylase polypeptide or polynucleotide, so as to increase or decrease proliferation of the cell. Decreased proliferation is useful for treating neoplastic cells, including both benign and malignant (cancer) cells. Increased proliferation may be desired, for example, to treat diseases characterized by low numbers of particular cell types, such as AIDS, or for increasing numbers of a cell population in vitro.

The invention provides assays for screening test compounds which bind to or modulate the activity of a mitochondrial deformylase polypeptide or a mitochondrial deformylase polynucleotide. A test compound preferably binds to a mitochondrial deformylase polypeptide or polynucleotide. More preferably, a test compound decreases a mitochondrial deformylase activity of a mitochondrial deformylase polypeptide or expression of a mitochondrial deformylase polynucleotide by at least about 10, preferably about 50, more preferably about 75, 90, or 100% relative to the absence of the test compound. Even more preferably, the test compound decreases or increases proliferation of a cell, such as a neoplastic cell, by at least about 10, preferably about 50, more preferably about 75, 90, or 100% relative to proliferation of the cell in the absence of the test compound.

### Test Compounds

Test compounds can be obtained using any of the numerous approaches in combinatorial library methods known in the art, including but not limited to, biological libraries, spatially addressable parallel solid phase or solution phase libraries, synthetic library methods requiring deconvolution, the ,,one-bead one-compound” library method, and synthetic library methods using affinity chromatography selection. The biological library approach is limited to polypeptide libraries, while the other four approaches are applicable to polypeptide, non-peptide oligomer or small molecule libraries of compounds. See Lam, Anticancer Drug Des. 12, 145, 1997.

Examples of methods for the synthesis of molecular libraries can be found in the art, for example in DeWitt et al., Proc. Natl. Acad. Sci. U.S.A. 90, 6909, 1993; Erb et al. Proc. Natl. Acad Sci. U.S.A. 91, 11422, 1994; Zuckermann et al., J. Med. Chem. 37, 2678, 1994; Cho et al., Science 261, 1303, 1993; Carell et al., Angew. Chem. Int. Ed. Engl. 33, 2059, 1994; Carell et al., Angew. Chem. Int. Ed. Engl. 33, 2061; and in Gallop et al., J. Med. Chem. 37, 1233, 1994.

Libraries of compounds may be presented in solution (see, e.g., Houghten, Biotechniques 13, 412-421, 1992), or on beads (Lam, Nature 354, 82-84, 1991), chips (Fodor, Nature 364, 555-556, 1993), bacteria or spores (Ladner, U.S. Pat. No. 5,223,409), plasmids (Cull et al., Proc. Natl. Acad. Sci. U.S.A. 89, 1865-1869, 1992) or on phage (Scott & Smith, Science 249, 386-390, 1990; Devlin, Science 249, 404-406, 1990); Cwirla et al., Proc. Natl. Acad. Sci. 97, 6378-6382, 1990; Felici, J. Mol. Biol. 222, 301-310,1991; and Ladner, U.S. Pat. No. 5,223,409).

### High Throughput Screening

Test compounds are preferably screened for the ability to bind to mitochondrial deformylase polypeptides or polynucleotides or to affect mitochondrial deformylase activity or mitochondrial deformylase gene expression using high throughput screening. Using high throughput screening, many discrete compounds can be tested in parallel so that large numbers of test compounds can be quickly screened. The most widely established techniques utilize 96-well microtiter plates. The wells of the microtiter plates typically require assay volumes that range from 50 to 500 ml. In addition to the plates, many instruments, materials, pipettors, robotics, plate washers and plate readers are commercially available to fit the 96-well format to a wide range of homogeneous and heterogeneous assays.

Alternatively, ,,free format assays,” or assays that have no physical barrier between samples, can be used. For example, an assay using pigment cells (melanocytes) in a simple homogeneous assay for combinatorial peptide libraries is described by Jayawickreme et al., Proc. Natl. Acad. Sci. U.S.A. 19, 1614-18 (1994). The cells are placed under agarose in petri dishes, then beads that carry combinatorial compounds are placed on the surface of the agarose. The combinatorial compounds are partially released the compounds from the beads. Active compounds can be visualized as dark pigment areas because, as the compounds diffuse locally into the gel matrix, the active compounds cause the cells to change colors.

Another example of a free format assay is described by Chelsky, “Strategies for Screening Combinatorial Libraries: Novel and Traditional Approaches,” reported at the First Annual Conference of The Society for Biomolecular Screening in Philadephia, Pa. (Nov. 7-10, 1995). Chelsky placed a simple homogenous enzyme assay for carbonic anhydrase inside an agarose gel such that the enzyme in the gel would cause a color change throughout the gel. Thereafter, beads carrying combinatorial compounds via a photolinker were placed inside the gel and the compounds were partially released by UV-light. Compounds that inhibited the enzyme were observed as local zones of inhibition having less color change.

Yet another example is described by Salmon et al., Molecular Diversity 2, 57-63 (1996). In this example, combinatorial libraries were screened for compounds that had cytotoxic effects on cancer cells growing in agar.

Another high throughput screening method is described in Beutel et al., U.S. Pat. No. 5,976,813. In this method, test samples are placed in a porous matrix. One or more assay components are then placed within, on top of, or at the bottom of a matrix such as a gel, a plastic sheet, a filter or other forms of easily manipulated solid support. When samples are introduced to the porous matrix they diffuse sufficiently slowly such that the assays can be performed without the test samples running together.

### Binding Assays

For binding assays, the test compound is preferably a small molecule which binds to and occupies the active site of the mitochondrial deformylase polypeptide thereby making the active site inaccessible to substrate such that normal biological activity is prevented. Examples of such small molecules include but are not limited to small peptides or peptide-like molecules. In binding assays, either the test compound or the target polypeptide can comprise a detectable label, such as a fluorescent, radioisotopic, or enzymatic label, such as horseradish peroxidase, alkaline phosphatase, or luciferase. Detection of a test compound which is bound to the target polypeptide can then be accomplished, for example, by direct counting of radioemmission or by scintillation counting, or by determination of conversion of an appropriate substrate to product.

Alternatively, binding of a test compound to a target polypeptide can be determined without labeling either of the interactants. For example, a microphysiometer can be used to detect binding of a test compound with the target polypeptide. A microphysiometer (e.g., CytosensorÔ) is an analytical instrument that measures the rate at which a cell acidifies its environment using a light-addressable potentiometric sensor (LAPS). Changes in this acidification rate can be used as an indicator of the interaction between a test compound and a target polypeptide. (McConnell et al., Science 257, 1906-1912, 1992).

In yet another aspect of the invention, a mitochondrial deformylase polypeptide can be used as a “bait protein” in a two-hybrid assay or three-hybrid assay (see, e.g., U.S. Pat. No. 5,283,317; Zervos et al., Cell 72, 223-232, 1993; Madura et al., J. Biol. Chem. 268, 12046-12054, 1993; Bartel et al., Biotechniques 14, 920-924, 1993; Iwabuchi et al., Oncogene 8, 1693-1696, 1993; and Brent W094/10300), to identify other proteins (captured proteins) which bind to or interact with the mitochondrial deformylase polypeptide and modulate its activity

The two-hybrid system is based on the modular nature of most transcription factors, which consist of separable DNA-binding and activation domains. Briefly, the assay utilizes two different DNA constructs. In one construct, the gene that codes for a protein of the invention is fused to a gene encoding the DNA binding domain of a known transcription factor (e.g., GAL4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact in vivo to form an protein-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected, and cell colonies containing the functional transcription factor can be isolated and used to obtain the cloned gene which encodes the protein which interacts with the mitochondrial deformylase polypeptide.

Determining the ability of a test compound to bind to a mitochondrial deformylase polypeptide can also be accomplished using a technology such as real-time Bimolecular Interaction Analysis (BIA). Sjolander & Urbaniczky, Anal. Chem. 63, 2338-2345, 1991, and Szabo et al., Curr. Opin. Struct. Biol. 5, 699-705, 1995. BIA is a technology for studying biospecific interactions in real time, without labeling any of the interactants (e.g., BIAcore™). Changes in the optical phenomenon surface plasmon resonance (SPR) can be used as an indication of real-time reactions between biological molecules.

It may be desirable to immobilize either the mitochondrial deformylase polypeptide or the test compound to facilitate separation of bound from unbound forms of one or both of the interactants, as well as to accommodate automation of the assay. Thus, either the mitochondrial deformylase polypeptide or the test compound is bound to a solid support. Suitable solid supports include, but are not limited to, glass or plastic slides, tissue culture plates, microtiter wells, tubes, silicon chips, or particles such as beads, including but not limited to latex, polystyrene, or glass beads. Any method known in the art can be used to attach the mitochondrial deformylase polypeptide or test compound to a solid support, including use of covalent and non-covalent linkages, passive absorption, or pairs of binding moieties attached respectively to the polynucleotide and the solid support. Test compounds are preferably bound to the solid support in an array, so that the location of individual test compounds can be tracked. Binding of a test compound to a mitochondrial deformylase polypeptide can be accomplished in any vessel suitable for containing the reactants. Examples of such vessels include microtiter plates, test tubes, and microcentrifuge tubes.

In one embodiment, the mitochondrial deformylase polypeptide is a fusion protein comprising a domain that allows the mitochondrial deformylase polypeptide to be bound to a solid support. For example, glutathione-S-transferase fusion proteins can be adsorbed onto glutathione sepharose beads (Sigma Chemical, St. Louis, Mo.) or glutathione derivatized microtiter plates, which are then combined with the test compound or the test compound and the non-adsorbed mitochondrial deformylase polypeptide, and the mixture incubated under conditions conducive to complex formation (e.g., at physiological conditions for salt and pH). Following incubation, the beads or microtiter plate wells are washed to remove any unbound components and binding of the interactants is determined either directly or indirectly, for example, as described above. Alternatively, the complexes can be dissociated from the solid support before binding is determined.

Other techniques for immobilizing proteins on a solid support also can be used in the screening assays of the invention. For example, either a mitochondrial deformylase polypeptide or a test compound can be immobilized utilizing conjugation of biotin and streptavidin. Biotinylated mitochondrial deformylase polypeptides or test compounds can be prepared from biotin-NHS(N-hydroxysuccinimide) using techniques well known in the art (e.g., biotinylation kit, Pierce Chemicals, Rockford, Ill.) and immobilized in the wells of streptavidin-coated 96 well plates (Pierce Chemical). Alternatively, antibodies which specifically bind to a mitochondrial deformylase polypeptide or a test compound, but which do not interfere with a desired binding site, such as the active site of the mitochondrial deformylase polypeptide, can be derivatized to the wells of the plate, and unbound target or protein trapped in the wells by antibody conjugation.

Methods for detecting such complexes, in addition to those described above for the GST-immobilized complexes, include immunodetection of complexes using antibodies which specifically bind to the mitochondrial deformylase polypeptide or test compound, as well as enzyme-linked assays which rely on detecting a mitochondrial deformylase activity of the mitochondrial deformylase polypeptide.

Screening for test compounds which bind to a mitochondrial deformylase polypeptide also can be carried out in an intact cell. Any cell which expresses a mitochondrial deformylase polynucleotide can be used in a cell-based assay system. The mitochondrial deformylase polynucleotide can be naturally occurring in the cell or can be introduced using techniques such as those described above. Either a primary culture or an established cell line, including neoplastic cell lines such as the colon cancer cell lines HCT116, DLD1, HT29, Caco2, SW837, SW480, and RKO, breast cancer cell lines 21-PT, 21-MT, MDA-468, SK-BR3, and BT-474, the A549 lung cancer cell line, and the H392 glioblastoma cell line, can be used. An intact cell is contacted with a test compound. Binding of the test compound to a mitochondrial deformylase polypeptide is determined as described above, after lysing the cell to release the mitochondrial deformylase polypeptide-test compound complex.

### Mitochondrial Deformylase Assays

Test compounds also can be tested for the ability to increase or decrease a mitochondrial deformylase activity of a mitochondrial deformylase polypeptide. Mitochondrial deformylase activity is preferably measured using the method described in Adams, J. Mol. Biol. 33, 571-89, as modified by Meinnel & Blanquet, J. Bacteriol. 177, 1883-87 (1995), or WO 99/57097 (see also Examples 2 and 5, below). Mitochondrial deformylase activity can be measured after contacting either a purified mitochondrial deformylase polypeptide, a cell extract, or an intact cell with a test compound. A test compound which decreases mitochondrial deformylase activity by at least about 10, preferably about 50, more preferably about 75, 90, or 100% is identified as a potential agent for decreasing cell proliferation. A test compound which increases mitochondrial deformylase activity by at least about 10, preferably about 50, more preferably about 75, 90, or 100% is identified as a potential agent for increasing cell proliferation.

### Mitochondrial Deformylase Gene Expression

In another embodiment, test compounds which increase or decrease mitochondrial deformylase gene expression are identified. A mitochondrial deformylase polynucleotide is contacted with a candidate compound and the expression of an RNA or protein product of the mitochondrial deformylase polynucleotide is determined. The level of expression of appropriate mRNA or protein in the presence of the candidate compound is compared to the level of expression of mRNA or protein in the absence of the candidate compound. The candidate compound can then be identified as a modulator of expression based on this comparison. For example, when expression of mRNA or protein is greater in the presence of the candidate compound than in its absence, the candidate compound is identified as a stimulator or enhancer of the mRNA or protein expression. Alternatively, when expression of the mRNA or protein is less in the presence of the candidate compound than in its absence, the candidate compound is identified as an inhibitor of the mRNA or protein expression.

The level of mRNA or protein expression in the cells can be determined by methods well known in the art for detecting mRNA or protein. Either qualitative or quantitative methods can be used. The presence of protein products of the disclosed genes can be determined, for example, using a variety of techniques known to the art, including immunochemical methods such as radioimmunoassay, Western blotting, and immunohistochemistry. Alternatively, protein synthesis can be determined in vivo, in a cell culture, or in an in vitro translation system by detecting incorporation of labeled amino acids into a mitochondrial deformylase polypeptide.

Such screening can be carried out either in a cell-free assay system or in an intact cell. Any cell which expresses a mitochondrial deformylase polynucleotide can be used in a cell-based assay system. The mitochondrial deformylase polynucleotide can be naturally occurring in the cell or can be introduced using techniques such as those described above. Either a primary culture or an established cell line, including neoplastic cell lines such as the colon cancer cell lines HCT116, DLD1, HT29, Caco2, SW837, SW480, and RKO, breast cancer cell lines 21-PT, 21-MT, MDA-468, SK-BR3, and BT-474, the A549 lung cancer cell line, and the H392 glioblastoma cell line, can be used.

### Pharmaceutical Compositions

An additional embodiment of the invention relates to the administration of a pharmaceutical composition, in conjunction with a pharmaceutically acceptable carrier, for any of the therapeutic effects discussed above. Such pharmaceutical compositions may comprise a mitochondrial deformylase polypeptide, mitochondrial deformylase polynucleotide, antibodies which specifically bind to a mitochondrial deformylase polypeptide, or mimetics, agonists, antagonists, or inhibitors of a mitochondrial deformylase polypeptide. In a preferred embodiment, the pharmaceutical composition comprises an antibody which specifically binds to a polypeptide comprising an amino acid sequence selected from the group consisting of amino acid sequences which are at least about 50% identical to the amino acid sequence shown in SEQ ID NO:2, the amino acid sequence shown in SEQ ID NO:2, amino acid sequences which are at least about 50% identical to the amino acid sequence shown in SEQ ID NO:4, the amino acid sequence shown in SEQ ID NO:4 amino acid sequences which are at least about 50% identical to the amino acid sequence shown in SEQ ID NO:6, and the amino acid sequence shown in SEQ ID NO:6. In a further preferred embodiment, the pharmaceutical composition comprises an antisense RNA or a ribozyme which is complementary to an RNA transcribed from a polynucleotide comprising a nucleotide sequence selected from the group consisting of nucleotide sequences which are at least about 50% identical to the nucleotide sequence shown in SEQ ID NO:1, the nucleotide sequence shown in SEQ ID NO:1, nucleotide sequences which are at least about 50% identical to the nucleotide sequence shown in SEQ ID NO:3, the nucleotide sequence shown in SEQ ID NO:3, nucleotide sequences which are at least about 50% identical to the nucleotide sequence shown in SEQ ID NO:5, and the nucleotide sequence shown in SEQ ID NO:5. In this context, the term ,,antisense RNA or a ribozyme” also comprises DNA sequences encoding said antisense RNA or ribozyme and which are, preferably, inserted in an expression vector which is useful for gene therapy. Such vectors are well known to the person skilled in the art.

The compositions may be administered alone or in combination with at least one other agent, such as stabilizing compound, which may be administered in any sterile, biocompatible pharmaceutical carrier, including, but not limited to, saline, buffered saline, dextrose, and water. The compositions may be administered to a patient alone, or in combination with other agents, drugs or hormones.

The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of REMINGTON'S PHARMACEUTICAL SCIENCES (Maack Publishing Co., Easton, Pa.). Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethylcellulose; gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers may also be used for delivery. Optionally, the suspension may also contain suitable stabilizers or agents which increase the solubility of the compounds to allow for the preparation of highly concentrated solutions. For topical or nasal administration, penetrants appropriate to the particular barrier to be permeated are used in the formulation. Such penetrants are generally known in the art.

The pharmaceutical compositions of the present invention may be manufactured in a manner that is known in the art, e.g., by means of conventional mixing, dissolving, granulating, dragee-making, levigating, emulsifying, encapsulating, entrapping, or lyophilizing processes. The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation may be a lyophilized powder which may contain any or all of the following: 1-50 mM histidine, 0.1%-2% sucrose, and 2-7% mannitol, at a pH range of 4.5 to 5.5, that is combined with buffer prior to use.

After pharmaceutical compositions have been prepared, they can be placed in an appropriate container and labeled for treatment of an indicated condition. Such labeling would include amount, frequency, and method of administration.

The determination of a therapeutically effective dose is well within the capability of those skilled in the art. A therapeutically effective dose refers to that amount of active ingredient which increases or decreases cell proliferation relative to cell proliferation which occurs in the absence of the therapeutically effective dose. Cell proliferation can be measured, inter alia, by counting dividing cells microscopically or by measuring the incorporation of ³H-thymidine.

For any compound, the therapeutically effective dose can be estimated initially either in cell culture assays or in animal models, usually mice, rabbits, dogs, or pigs. The animal model may also be used to determine the appropriate concentration range and route of administration. Such information can then be used to determine useful doses and routes for administration in humans.

Therapeutic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio of toxic to therapeutic effects is the therapeutic index, and it can be expressed as the ratio, LD₅₀/ED₅₀.

Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED₅₀ with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active moiety or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

### Therapeutic Indications and Methods

This invention further pertains to the use of novel agents identified by the screening assays described above. Accordingly, it is within the scope of this invention to use a test compound identified as described herein in an appropriate animal model. For example, an agent identified as described herein (e.g., a modulating agent, an antisense nucleic acid molecule, a specific antibody, ribozyme, or a protein-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

The human mitochondrial deformylase gene provides a therapeutic target for decreasing cell proliferation, in particular for treating cancer or other diseases involving increased levels of cell proliferation. Cancers which can be treated according to the invention include adenocarcinoma, leukemia, lymphoma, melanoma, myeloma, sarcoma, and teratocarcinoma, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus. Other proliferative disorders, such as anhydric hereditary ectodermal dysplasia, congenital alveolar dysplasia, epithelial dysplasia of the cervix, fibrous dysplasia of bone, mammary dysplasia, and hyperplasias, for example, endometrial, adrenal, breast, prostate, or thyroid hyperplasias, or pseudoepitheliomatous hyperplasia of the skin, also can be treated with compositions.

The mitochondrial deformylase is of critical importance to both central and peripheral nervous system and is therefore a promising new target for the treatment of nervous system disease, for example in primary and secondary disorders after brain injury, disorders of mood, anxiety disorders, disorders of thought and volition, disorders of sleep and wakefulness, diseases of the motor unit like neurogenic and myopathic disorders, neurodegenerative disorders like Alzheimer's and Parkinson's disease, disorders leading to peripheral and chronic pain.

A reagent which affects mitochondrial deformylase activity can be administered to a human cell, either in vitro or in vivo, to reduce mitochondrial deformylase activity. The reagent preferably binds to an expression product of a human mitochondrial deformylase gene. If the expression product is a protein, the reagent is preferably a small molecule or an antibody. For treatment of human cells ex vivo an antibody can be added to a preparation of stem cells which have been removed from the body. The cells can then be replaced in the same or another human body, with or without clonal propagation, as is known in the art.

In one embodiment, the reagent is delivered using a liposome. Preferably, the liposome is stable in the animal into which it has been administered for at least about 30 minutes, more preferably for at least about 1 hour and even more preferably for at least about 24 hours. A liposome comprises a lipid composition that is capable of targeting a reagent, particularly a polynucleotide, to a particular site in an animal, such as a human. Preferably, the lipid composition of the liposome is capable of targeting to any organ of an animal, such as the lung, liver, spleen, heart brain, lymph nodes, and skin.

A liposome useful in the present invention comprises a lipid composition that is capable of fusing with the plasma membrane of the targeted cell to deliver its contents to the cell. Preferably, the transfection efficiency of a liposome is about 0.5 mg of DNA per 16 nmole of liposome delivered to about 10⁶ cells, more preferably about 1.0 mg of DNA per 16 nmol of liposome delivered to about 10⁶ cells, and even more preferably about 2.0 mg of DNA per 16 nmol of liposome delivered to about 10⁶ cells. Preferably, a liposome is between about 100 and 500 nm, more preferably between about 150 and 450 nm and even more preferably between about 200 and 400 nm in diameter.

Suitable liposomes for use in the present invention include those liposomes standardly used in, for example, gene delivery methods known to those of skill in the art. More preferred liposomes comprise liposomes having a polycationic lipid composition and/or liposomes having a cholesterol backbone conjugated to polyethylene glycol. Optionally, a liposome comprises a compound capable of targeting the liposome to a tumor cell. Such a liposome preferably includes a tumor cell ligand exposed on the outer surface of the liposome.

Complexing a liposome with a polynucleotide, such as an antisense oligonucleotide or ribozyme, can be achieved using methods which are standard in the art (see, for example, U.S. Pat. No. 5,705,151). Preferably, from about 0.1 mg to about 10 mg of polynucleotide is combined with about 8 nmol of liposomes, more preferably from about 0.5 mg to about 5 mg of polynucleotides are combined with about 8 nmol liposomes, and even more preferably about 1.0 mg of polynucleotides is combined with about 8 nmol liposomes.

In another embodiment, antibodies can be delivered to specific tissues in vivo using receptor-mediated targeted delivery. Receptor-mediated DNA delivery techniques are taught in, for example, Findeis et al. Trends in Biotechnol. 11, 202-05, (1993); Chiou et al., GENE THERAPEUTICS: METHODS AND APPLICATIONS OF DIRECT GENE TRANSFER (J. A. Wolff, ed.) (1994); Wu & Wu, J. Biol. Chem. 263, 621-24, 1988; Wu et al., J. Biol. Chem. 269, 542-46, 1994; Zenke et al., Proc. Natl. Acad. Sci. U.S.A. 87, 3655-59, 1990; Wu et al., J. Biol. Chem. 266, 338-42, 1991.

If single-chain antibodies are used, polynucleotides encoding the antibodies can be constructed and introduced into cells either ex vivo or in vivo using well-established techniques including, but not limited to, transferrin-polycation-mediated DNA transfer, transfection with naked or encapsulated nucleic acids, liposome-mediated cellular fusion, intracellular transportation of DNA-coated latex beads, protoplast fusion, viral infection, electroporation, ,,gene gun,” and DEAE- or calcium phosphate-mediated transfection.

Effective in vivo dosages of an antibody are in the range of about 5 mg to about 50 mg/kg, about 50 mg to about 5 mg/kg, about 100 mg to about 500 mg/kg of patient body weight, and about 200 to about 250 mg/kg of patient body weight. For administration of polynucleotides encoding single-chain antibodies, effective in vivo dosages are in the range of about 100 ng to about 200 ng, 500 ng to about 50 mg, about 1 mg to about 2 mg, about 5 mg to about 500 mg, and about 20 mg to about 100 mg of DNA.

If the expression product is mRNA, the reagent is preferably an antisense oligonucleotide or a ribozyme. Polynucleotides which express antisense oligonucleotides or ribozymes can be introduced into cells by a variety of methods, as described above.

Preferably, a reagent reduces expression of a mitochondrial deformylase gene or the activity of a mitochondrial deformylase polypeptide by at least about 10, preferably about 50, more preferably about 75, 90, or 100% relative to the absence of the reagent. The effectiveness of the mechanism chosen to decrease the level of expression of a mitochondrial deformylase gene or the activity of a mitochondrial deformylase polypeptide can be assessed using methods well known in the art, such as hybridization of nucleotide probes to mitochondrial deformylase-specific mRNA, quantitative RT-PCR, immunologic detection of a mitochondrial deformylase polypeptide, or measurement of mitochondrial deformylase activity.

Disorders characterized by lowered cell proliferation or a loss of specific cell types, such as Alzheimer's Disease, AIDS, muscular dystrophy, amyotrophic lateral sclerosis, or other muscle wasting diseases, autoimmune diseases, or a disease in which the cell is infected with a pathogen, such as a virus, bacterium, fungus, mycoplasm, or protozoan, can be treated with an agonist or activator of mitochondrial deformylase, to increase cell proliferation. Introduction of a mitochondrial deformylase polynucleotide which expresses a mitochondrial deformylase polypeptide, e.g. by gene therapy, also can be used to increase cell proliferation.

In any of the embodiments described above, any of the pharmaceutical compositions of the invention can be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

Any of the therapeutic methods described above may be applied to any subject in need of such therapy, including, for example, birds and mammals such as dogs, cats, cows, pigs, sheep, goats, horses, rabbits, monkeys, and most preferably, humans.

All documents cited in this disclosure are expressly incorporated herein. The above disclosure generally describes the present invention, and all references cited in this disclosure are incorporated by reference herein. A more complete understanding can be obtained by reference to the following specific examples which are provided for purposes of illustration only and are not intended to limit the scope of the invention.

### EXAMPLE 1

### Recombinant Expression of a DNA Sequence Encoding a Mitochondrial Deformylase Polypeptide in Yeast Cells (Pichia pastoris)

To produce large quantities of a mitochondrial deformylase polypeptides in yeast, the Pichia pastoris expression vector pPICZB (Invitrogen, San Diego, Calif.) is used. The mitochondrial deformylase polypeptide encoding DNA sequence is derived from the nucleotide sequence (SEQ ID NO:5) encoding the amino acid sequence (SEQ ID NO:6). Before insertion into vector pPICZB the DNA sequence is modified by well known methods in such a way that it contains at its 5′-end an initiation codon and at its 3′-end an enterokinase cleavage site, a His6 reporter tag and a termination codon. Moreover, at both termini recognition sequences for restriction endonucleases are added and after digestion of the multiple cloning site of pPICZ B with the corresponding restriciton enzymes the modified mitochondrial deformylase polypeptide encoding DNA sequence is ligated into pPICZB. This expression vector is designed for inducible expression in Pichia pastoris, expression is driven by a yeast promoter. The resulting pPICZ/md-His6 vector is used to transform the yeast. The yeast is cultivated under usual conditions in 5 1 shake flasks and the recombinantly produced protein isolated from the culture by affinity chromatography (Ni-NTA-Resin) in the presence of 8 M urea. The bound polypeptide is eluted with buffer, pH 3,5, and neutralized. Separation of the mitochondrial deformylase polypeptide from the His6 reporter tag is accomplished by site-specific proteolysis using enterokinase (Invitrogen, San Diego, Calif.) according to manufacturer's instructions. Purified mitochondrial deformylase polypeptide is obtained.

### EXAMPLE 2

### Determination of the Deformylase Activity of the Mitochondrial Deformylase Polypeptide Prepared According to Example 1

In this assay the dipeptide substrate, N-formyl-methionylleucyl-p-nitroaniline (f-ML-pNA) is first deformylated by the deformylase to give the corresponding dipeptide with a free amino terminus, which is a substrate for an aminopeptidase from Aeromonas proteolytica (Sigma Chemical Company). Sequential action by the aminopeptidase releases p-nitroaniline, a chromophore which can be detected spectrophotometrically. The dipeptide substrate is prepared as described in Wei and Pei 250, (1997), Anal. Biochem., 29-34. Assays are carried out at 23° C. in polystyrene cuvettes which contain 50 mM potassium phosphate, pH 7.0, 100 μM EGTA, 0 to 200 μM dipeptide substrate and 0.8 unit Aeromonas aminopeptidase. Reactions are initiated by addition of 10 to 100 μl (0.1 to 100 μg) of the mitochondrial deformylase polypeptide prepared according to Example 1, diluted in 50 mM HEPES, pH 7.0, containing 100 μg/ml BSA. Reactions are monitored continuously at 405 nm in a Perkin-Elmer λ3 UV/VIS spectrophotometer, and the initial rates are calculated from the early part of the reaction progression curves (<60s). Reactions at the lowest and highest dipeptide substrate concentration are generally repeated with doubled amount of the aminopeptidase (1.6 U) to insure that the deformylase reaction is rate-limiting in the coupled reaction sequence. The results obtained indicate that the mitochondrial deformylase polypeptide prepared according to Example 1 has deformylase activity.

### EXAMPLE 3

### Proliferation Inhibition Assay: Antisense Oligonucleotides Suppress the Growth of Cancer Cell Lines

The Cell line used for testing is the human colon cancer cell line HCT 116. Cells are cultured in RPMI-1640 with 10-15% fetal calf serum at a concentration of 10,000 cells per milliliter in a volume of 0.5 ml and kept at 37° C. in a 95% air/5% CO₂ atmosphere.

Phosphorothioate oligoribonucleotides are synthesized on an Applied Biosystems Model 380B DNA synthesizer using phosphoramidite chemistry. Two sequences of 24 bases are used: (1) 5′-CAG CGA TTT AAA TAC GGA ACA AGG-3′ (complementary to the nucleotides at position 1 to 24 of SEQ ID NO:1) and (2) 5′-AAA ATG CAG GTA AGC ATG TGA AAA-3′ (complementary to the nucleotides at position 1 to 24 of SEQ ID NO:3), as a control another (random) sequence (3) 5′-TCA ACT GAC TAG ATG TAC ATG GAC-3′ is used. Following assembly and deprotection, oligonucleotides are ethanol-precipitated twice, dried, and suspended in phosphate-buffered saline (PBS) at the desired concentration. Purity of these oligonucleotides is tested by capillary gel electrophoresis and ion exchange HPLC. The purified oligonucleotides are added to the culture medium at a concentration of 10 micromolar once per day for seven days.

The addition of oligonucleotides (1) and (2) for seven days results in significantly reduced expression of the mitochondrial deformylase as determined by Western blotting. This effect is not observed with oligonucleotide (3). After 3 to 7 days, the number of cells is counted using an automatic cell counter. The number of cells in cultures treated with oligonucleotide (3) (expressed at 100%) is compared with the number of cells in cultures treated with oligonucleotides (1) and (2), respectively. The number of cells in cultures treated with oligonucleotides (1) and (2) is not more than 30% of control, indicating that the inhibition of human mitochondrial deformylase has an anti-proliferative effect on cancer cells.

### EXAMPLE 4

### Proliferation Activation Assay: Cell Lines Transfected with the Mitochondrial Deformylase Gene Increase Growing Rates

Standard cell lines suitable for transfection and protein expression as known in the art are cultured under appropriate conditions as described by the suppliers of cell lines, e.g. the ATCC.

Cells are transfected with expression constructs for the mitochondrial deformylase gene as known in the art. The expression of the protein is induced as known in the art. After 3 to 7 days, the number of cells is counted using an automatic cell counter. The number of cells in cultures with transfected cells is compared with the number of cells in cultures with untransfected cells. The number of cells in cultures which are transfected with the mitochondrial deformylase is more than 30% of control, indicating that the augmentation of human mitochondrial deformylase activity has a proliferative effect on cells.

### EXAMPLE 5

### Identification of a Test Compound which Binds to a Mitochondrial Deformylase Polypeptide

Purified mitochondrial deformylase polypeptides comprising a glutathione-S-transferase protein and absorbed onto glutathione-derivatized wells of 96-well microtiter plates are contacted with test compounds from a small molecule library at pH 7.0 in a physiological buffer solution. The mitochondrial deformylase polypeptide comprises an amino acid sequence shown in SEQ ID NO:2, 4 or 6. The test compounds comprise a fluorescent tag. The samples are incubated for 5 minutes to one hour. Control samples are incubated in the absence of a test compound.

The buffer solution containing the test compounds is washed from the wells. Binding of a test compound to a mitochondrial deformylase polypeptide is detected by fluorescence measurements of the contents of the wells. A test compound which increases the fluorescence in a well by at least 15% relative to fluorescence of a well in which a test compound was not incubated is identified as a compound which binds to a mitochondrial deformylase polypeptide.

### EXAMPLE 6

### Identification of a Test Compound which Decreases Mitochondrial Deformylase Activity

Extracts from the human colon cancer cell line HCT116 are contacted with test compounds from a small molecule library in 50 ml of 50 mM HEPES, pH 7.0, and 0.5 M KCl and assayed for mitochondrial deformylase activity as described in Adams (1968), as modified by Meinnel & Blanquet, 1995. Control extracts, in the absence of a test compound, also are assayed. Mitochondrial deformylase activity of the extracts is measured in the presence of 4 mM substrate (Fo-Met-Ala-Ser; Sigma). After incubation at for 5 minutes to 1 hour at 37° C. in 50 mM HEPES (N-2-hydroxyethylpiperazine-N′-2-ethanesulfonic acid sodium salt)-HCl, pH 7.0, and 0.5 M KCl for five minutes, the reaction is stopped with 500 ml of trichlorocetic acid. The samples are centrifuged briefly to remove protein, and 500 ml of supernatant is removed and added to 500 ml of 0.2 M KOH and 0.25 M potassium acetate buffer, pH 5.2

A ninhydrin assay according to Moore & Stein, J. Biol. Chem. 211, 907 (1954) is then performed. The color which develops is measured at 350 mm. A test compound which decreases mitochondrial deformylase activity of the extract relative to the control extract by at least 20% is identified as a mitochondrial deformylase inhibitor. Alternatively, determination of deformylase activity is carried out as described in Example 2, above.

### EXAMPLE 7

### Identification of a Test Compound which Decreases Mitochondrial Deformylase Gene Expression

A test compound is administered to a culture of the breast tumor cell line MDA-468 and incubated at 37° C. for 10 to 45 minutes. A culture of the same type of cells incubated for the same time without the test compound provides a negative control.

RNA is isolated from the two cultures as described in Chirgwin et al., Biochem. 18, 5294-99, 1979). Northern blots are prepared using 20 to 30 μg total RNA and hybridized with a ³²P-labeled mitochondrial deformylase-specific probe at 65° C. in Express-hyb (ClonTech). The probe comprises at least 11 contiguous nucleotides selected from SEQ ID NOS:1, 3 or 5. A test compound which decreases the mitochondrial deformylase-specific signal relative to the signal obtained in the absence of the test compound is identified as an inhibitor of mitochondrial deformylase gene expression.

### EXAMPLE 8

Treatment of a Breast Tumor with a Reagent which Specifically Binds to a Mitochondrial Deformylase Gene Product

Synthesis of antisense mitochondrial deformylase oligonucleotides comprising at least 11 contiguous nucleotide selected from SEQ ID NOS:1, 3 or 5 is performed on a Pharmacia Gene Assembler series synthesizer using the phosphoramidite procedure (Uhlmann et al., Chem. Rev. 90, 534-83, 1990). Following assembly and deprotection, oligonucleotides are ethanol-precipitated twice, dried, and suspended in phosphate-buffered saline (PBS) at the desired concentration. Purity of these oligonucleotides is tested by capillary gel electrophoreses and ion exchange HPLC. Endotoxin levels in the oligonucleotide preparation are determined using the Luminous Amebocyte Assay (Bang, Biol. Bull. (Woods Hole, Mass.) 105, 361-362, 1953).

The antisense oligonucleotides are injected directly into the breast tumor in an aqueous medium (an aqueous composition) at a concentration of 0.1-100 mM with a needle. The needle is placed in the tumors and withdrawn while expressing the aqueous composition within the tumor.

The size of the breast tumor is monitored over a period of days or weeks. Additional injections of the antisense oligonucleotides may be given during that time. The size of the breast tumor gradually decreases due to decreased proliferation of the breast tumor cells.

1. A substantially purified human mitochondrial deformylase
a) consisting essentially of the amino acid sequence of SEQ ID NO:6 and fragments thereof; or
b) of an amino acid sequence wherein a substitution, deletion, addition or transpostion of one to several amino acid residue(s) is made in SEQ ID NO:6.

2. An isolated and purified polynucleotide encoding the mitochondrial deformylase of claim 1, a degenerate variant thereof, or a nucleotide sequence which is complementary thereto.

3. An isolated and purified polynucleotide consisting essentially of the sequence of SEQ ID NO:5, a conservative substitution variant thereof, active fragments, or functional equivalents thereof, or a nucleotide sequence which is complementary thereto.

4. An isolated and purified cDNA or RNA comprising of the sequence of SEQ ID NO:5, a conservative substitution variant thereof, active fragments, or functional equivalents thereof, or a nucleotide sequence which is complementary thereto.

5. A polynucleotide which hybridizes under stringent conditions to the polynucleotide sequence of claim 2.

6. A hybridization probe comprising at least 10 contiguous nucleotides selected from SEQ ID NO:2.

7. An expression vector containing the polynucleotide sequence of claim 2.

8. A host cell containing the expression vector of claim 6.

9. A method for producing a polypeptide comprising SEQ ID NO:6, the method comprising the steps of:
a) culturing the host cell of claim 6 under conditions suitable for the expression of the polypeptide; and
b) recovering the polypeptide from the host cell culture.

10. A method for detection of polynucleotides in a biological sample comprising the steps of:
a) hybrizing a polynucleotide consisting of SEQ ID NO:1, SEQ ID NO:3 or SEQ ID:5 or a complement of any of them to nucleic acid material of a biological sample, thereby forming a hybridization complex; and
b) detecting said hybridization complex; wherein the presence of said complex correlates with the presence of the polynucleotide consiting of SEQ ID NOS:1, 3 and 5 in said biological sample.

11. The method of claim 9 wherein before hybridization, the nucleic acid material of the biological sample is amplified.

12. A method for the detection of an expression product of the polynucleotide of SEQ ID NOS:1, 3 or 5 comprising the steps of
contacting a biological sample with a reagent which specifically interacts with the expression product and
detecting the interaction.

13. The expression product of claim 11 being a polynucleotide.

14. The expression product of claim 11 being a peptide.

15. A diagnostic kit comprising the reagents of one of the claims 9, 10 or 11.

16. A method of screening for agents which can regulate the activity of human mitochondrial deformylase, comprising the steps of:
contacting a test compound with a polypeptide comprising an amino acid sequence which is at least about 52% identical to the amino acid sequence shown in SEQ ID NOS:2; 4 or 6 and
detecting binding of the test compound to the polypeptide, wherein a test compound which binds to the polypeptide is identified as a potential therapeutic agent for regulating the activity of human mitochondrial deformylase.

17. The method of claim 14 wherein the step of contacting is in a cell.

18. The method of claim 15 wherein the cell is in vitro.

19. The method of claim 14 wherein the step of contacting is in a cell-free system.

20. The method of claim 14 wherein the polypeptide comprises a detectable label.

21. The method of claim 14 wherein the test compound comprises a detectable label.

22. The method of claim 14 wherein the test compound displaces a labeled ligand which is bound to the polypeptide.

23. The method of claim 14 wherein the polypeptide is bound to a solid support.

24. The method of claim 14 wherein the test compound is bound to a solid support.

25. A method of screening for agents which regulate an activity of human mitochondrial deformylase, comprising the steps of:
contacting a test compound with a polypeptide comprising an amino acid sequence which is at least about 52% identical to the amino acid sequences shown in SEQ ID NOS:2, 4 or 6; and
detecting a mitochondrial deformylase activity of the polypeptide, wherein a test compound which increases the mitochondrial deformylase activity is identified as a potential therapeutic agent for increasing the activity of the human mitochondrial deformylase, and wherein a test compound which decreases the mitochondrial deformylase activity of the polypeptide is identified as a potential therapeutic agent for decreasing the activity of the human mitochondrial deformylase.

26. The method of claim 23 wherein the step of contacting is in a cell.

27. The method of claim 24 wherein the cell is in vitro.

28. The method of claim 23 wherein the step of contacting is in a cell-free system.

29. A method of screening for agents which regulate an activity of human mitochondrial deformylase, comprising the steps of:
contacting a test compound with a product encoded by a polynucleotide which comprises a nucleotide sequence which is at least about 50% identical to the nucleotide sequences shown in SEQ ID NOS:1, 3 or 5 or complements theretof; and
detecting binding of the test compound to the product, wherein a test compound which binds to the product is identified as a potential therapeutic agent for regulating the activity of human mitochondrial deformylase.

30. The method of claim 27 wherein the product is a polypeptide.

31. The method of claim 27 wherein the product is RNA.

32. A method of reducing activity of human mitochondrial deformylase, comprising the step of:
contacting a cell with a reagent which specifically binds to a product encoded by a polynucleotide comprising a nucleotide sequence which is at least about 50% identical to the nucleotide sequences shown in SEQ ID NOS:1, 3 or 5, whereby the activity of human mitochondrial deformylase is reduced.

33. The method of claim 30 wherein the product is a polypeptide.

34. The method of claim 31 wherein the reagent is an antibody.

35. The method of claim 30 wherein the product is RNA.

36. The method of claim 33 wherein the reagent is an antisense oligonucleotide.

37. The method of claim 33 wherein the reagent is a ribozyme.

38. The method of claim 30 wherein the cell is in vitro.

39. The method of claim 30 wherein the cell is in vivo.

40. A purified reagent that specifically binds to and modulates activity of the polypeptide of claim 1, wherein said reagent is identified by the method of claim 14 or 23.

41. A pharmaceutical composition, comprising:
a reagent which specifically binds to a product encoded by a polynucleotide comprising a nucleotide sequence which is at least about 50% identical to the nucleotide sequences shown in SEQ ID NOS:1, 3 or 5, wherein said reagent is identified by the method of claim 27 or 30; and
a pharmaceutically acceptable carrier.

42. The pharmaceutical composition of claim 38, wherein the reagent is an antibody.

43. The pharmaceutical composition of claim 38, wherein the reagent is an antisense oligonucleotide.

44. The pharmaceutical composition of claim 38, wherein the reagent is a ribozyme.

45. A pharmaceutical composition, comprising:
an expression construct encoding a polypeptide comprising the amino acid sequences shown in SEQ ID NOS:2, 4 or 6; and
a pharmaceutically acceptable carrier.

46. A method for treating neoplastic disease comprising administering to a subject in need of such treatment an effective amount of the reagent of claim 38 or 39.

47. A preparation of antibodies which specifically binds to a polypeptide consisting essentially of the amino acid sequences shown in SEQ ID NOS:2, 4 or 6.

48. The preparation of claim 46 wherein the antibodies are polyclonal. The preparation of claim 46 wherein the antibodies are monoclonal.
